(19) United States
(12) Patent Application Publication
(10) Pub. No.: US 2025/0325473 A1
(43) Pub. Date: Oct. 23, 2025

(54) COLLAGEN-IMPREGNATED DEVICES AND METHODS FOR TREATMENT OF CANCER

(71) Applicant: COLLAGEN MATRIX, INC., Oakland, NJ (US)

(72) Inventors: Daniel Ammon, Webster, NY (US); Hui-Chen Chen, Wayne, NJ (US); Diana Legarda, Fair Lawn, NJ (US); Joseph Chinn, Lafayette, CO (US)

(21) Appl. No.: 18/871,905

(22) PCT Filed: Jun. 5, 2023
(86) PCT No.: PCT/US2023/024495

§ 371 Date: Dec. 5, 2024

Related U.S. Application Data
(60) Provisional application No. 63/349,300, filed on Jun. 6, 2022.

(51) Int. Cl. A61K 9/00; A61K 38/08; A61K 47/42

(52) U.S. Cl. CPC A61K 9/0024; A61K 38/08; A61K 47/42

(57) ABSTRACT

Collagen devices and methods of treating cancer in a patient. Such methods include the step of introducing a collagen device having a DDR1 peptide into the patient, whereby the DDR1 peptide is delivered to the cancer. Other such methods include the step of introducing a DDR1 peptide into the patient, whereby the DDR1 peptide is delivered to the cancer. These methods may also include the step of administering an anti-cancer therapeutic agent to the patient.

### RELATED APPLICATIONS

This application is a 371 national stage entry of International Application No. PCT/US2023/24495, filed Jun. 5, 2023, which claims priority to U.S. Provisional Patent Application Ser. No. 63/349,300, filed Jun. 6, 2022, the disclosure of which is hereby incorporated by reference in its entirety.

### FIELD OF THE INVENTION

Embodiments of the invention pertain to customizable collagen intended for application to various tissues for diverse purposes, including cancer treatment. More specifically, embodiments of the invention relate to methods for designing a device to release peptides over time to treat cancer. The methods may include the amplification or reduction of certain characteristics of collagen for targeted uses (e.g., as described in Applicant's International Patent Application No. PCT/US2021/049646, published as WO 2022/060622 A1, the disclosure of which is incorporated herein by reference), such as for these cancer applications. Embodiments of the invention also relate to engineered collagen constructs, including layered targeted collagen products/constructs, methods for making same, methods using 3D printing of biomolecules including the customizable collagen, and collagen constructs formed by such methods. Embodiments also include injectable forms and bandage designs. Further embodiments include one or more binding active pharmaceutical ingredients (APIs) to cell binding peptides, and engineering or inducing cancer-fighting immune cells, such as T cells, to express DDR1 peptide that allow them to prevent collagen alignment.

### SEQUENCE LISTING

The instant application contains a Sequence Listing which has been submitted via Patent Center and is hereby incorporated by reference in its entirety. Said.xml copy, created on Jul. 8, 2025, is named 1318-005US01-Sequence-Listing, and is 73,316 bytes in size.

### BACKGROUND OF THE INVENTION

Collagen is a naturally occurring protein found in humans and animals. Collagen tissue is often procured from a donor and used in a wide variety of medical applications, ranging from cosmetic surgery to bone repair to wound healing. Prokaryote collagen can also be derived from genetically engineered microorganisms.

Collagen may be treated and/or processed in several different ways such as being augmented, reconstituted, concentrated, cross-linked, combined with other biological substances, and so on. As such, various collagen products may be produced for diverse medical applications. Some embodiments of the present invention relate generally to rapid prototyping systems, specifically, 3D printing systems for making collagen based medical and dental devices such as, for example, dental bone grafting, dental membranes, stents, punctal plugs, ocular collagen onlays and inlays, contact lenses, orthopedic bone application, spine application, nerve applications and skin applications. Various embodiments/processes of the invention relate to the use of ink-jet printing, fused deposition modeling (FDM), selective laser sintering (SLS), stereolithography (SLA), digital light processing (DLP), bio-printing or combinations thereof to build-up the medical devices as three-dimensional objects from many material systems and novel resin systems of this invention. Ink-jet printing system dispenses materials through ink-jet printing head to form 3D objects, which harden by cooling, polymerization, and light irradiation. FDM extrudes thermoplastic materials throughout nozzle to build 3D object. SLS uses laser as power source to sinter powdered materials to form solid objects. SLA using laser beam traces out the shape of each layer and hardens the photosensitive resin in a vat (reservoir or bath). DLP system builds three-dimensional objects by using the Digital Light Processor (DLP) projector to project sequential voxel planes into liquid resin, which then caused the liquid resin to cure. Bioprinting is a layer-by-layer process is which a biological matrix is printed either with or without cells. The objects can then act as a matrix or scaffold to grow cellularized tissue. In general, rapid prototyping refers to a conventional manufacturing process used to make parts, wherein the part is built on a layer-by-layer basis using layers of hardening material. Per this technology, the part to be manufactured is considered a series of discrete cross-sectional regions which, when combined, make-up a three-dimensional structure. The building-up of a part layer-by-layer is very different than conventional machining technologies, where metal or plastic pieces are cut and drilled to a desired shape. In rapid prototyping technology, the parts are produced directly from computer-aided design (CAD) or other digital images. Software is used to slice the digital image into thin cross-sectional layers. Then, the part is constructed by placing layers of plastic or other hardening material on top of each other. There are many different techniques that can be used to combine the layers of structural material. A final curing step may be required to fully cure the layers of material for some of the techniques. The application of sealer may be needed to form a dense 3D object for some of the techniques, such as inkjet printing of a powder bed or FDM. Additional milling may be added to some of the techniques.

Ink-jet printing technology is a rapid prototyping method that can be used to fabricate the three-dimensional object. In one well known ink-jet printing method that was developed at Massachusetts Institute of Technology, as described in Sachs et al., U.S. Pat. No. 5,204,055 (incorporated by reference herein in its entirety), printer heads are used to discharge a binder material onto a layer of powder particulate in a powder bed. The powdered layer corresponds to a digitally superposed section of the object that will be produced. The binder causes the powder particles to fuse together in selected areas. This results in a fused cross-sectional segment of the object being formed on the platform. The steps are repeated for each new layer until the desired object is achieved. In a final step, a laser beam scans the object causing the powdered layers to sinter and fuse together if needed. In another ink-jet printing process, as described in Sanders, U.S. Pat. Nos. 5,506,607 and 5,740,051, a low-melting thermoplastic material is dispensed through one ink-jet printing head to form a three-dimensional object. A second ink-jet printer head dispenses wax material or other supporting material to form supports for the three-dimensional object. After the object has been produced, the wax supports are removed, and the object is finished as needed. MultiJet printers, such as, the high-quality PolyJet and MultiJet 3D printing processes use a UV light to crosslink a photopolymer. However, rather than scanning a laser to cure layers, a printer jet sprays tiny droplets of the photopolymer (similar to ink in an inkjet printer) in the shape of the first layer. The UV lamp attached to the printer head crosslinks the polymer and locks the shape of the layer in place. The build platform then descends by one layer thickness, and more material is deposited directly onto the previous layer. Triple-jetting technology (PolyJet) used in Stratasys Objet 500 Connex3, is the most advanced method of PolyJet 3D printing. This technology performs precise printing with three materials and thus makes three-color mixing possible.

Fused deposition modeling (FDM) technology was developed and implemented at first time by Scott Crump, Stratasys Ltd. founder, in 1980s. What is good about this technology that all parts printed with FDM can go in high-performance and engineering-grade thermoplastic. FDM is the only 3D printing technology that builds parts with production-grade thermoplastics, so things printed are of excellent mechanical, thermal and chemical qualities. 3D printing machines that use FDM Technology build objects layer by layer from the bottom up by heating and extruding thermoplastic filament. Along to thermoplastic a printer can extrude support materials as well. Then the printer heats thermoplastic till its melting point and extrudes it throughout nozzle to a build platform. To support upper layer the printer may place underneath special material that can be dissolved after printing is completed. When the thin layer of plastic binds to the layer beneath it, it cools down and hardens. Once the layer is finished, the base is lowered to start building of the next layer. This technology is considered simple-to-use and environment-friendly. Different kind of thermoplastics can be used to print dental objects.

Selective Laser Sintering (SLS) is a technique that uses laser as power source to form solid 3D objects. This technique was developed by Carl Deckard, a student of Texas University, and his professor Joe Beaman in 1980s. The main difference between SLS and SLA is that it uses powdered material in the vat instead of liquid resin as stereolithography does. Unlike some other additive manufacturing processes, such as stereolithography (SLA) and fused deposition modeling (FDM), SLS doesn't need to use any support structures as the object being printed is constantly surrounded by unsintered powder. Due to wide variety of materials that can be used with this type of 3D printer the technology is very popular for 3D printing customized products. SLS requires the use of high-powered lasers, which makes the printer to be very expensive. Extensive surface finishing is required for dental objects made with this process.

SLA 3D printing method was patented by Charles Hull, co-founder of 3D Systems, Inc. in 1986, which converts liquid plastic into solid 3D objects. SLA 3D printers work with excess of liquid resin that hardens and forms into solid object by irradiation. Parts built usually have smooth surfaces, but their quality varies depending on the quality of SLA machine used. After plastic hardens a platform of the printer drops down (top-down printer) or moves up (bottom-up printer) in the tank a fraction of a millimeter and laser-forms the next layer until printing is completed. Once all layers are printed, the object is rinsed with a solvent and then placed in a post-cure oven to finish processing.

Digital Light Processing is another 3D Printing process very similar to stereolithography. The DLP technology was created in 1987 by Larry Hornbeck of Texas Instruments and became very popular in Projectors production. It uses digital micro mirrors laid out on a semiconductor chip. 3D inkjet, DLP and SLA all works with photopolymers. The difference between SLA and DLP processes is a different light source. DLP method projects sequential voxel planes into liquid resin, which then caused the liquid resin to cure. The material used for printing is liquid resin that is placed in the transparent resin container. The resin hardens quickly when affected by irradiation of light. The printing speed is impressive, especially with Carbon3D′s CLIP (Continuous Liquid Interface Production) technology. The layer of hardened material can be created with such printer in a few seconds. When the layer is finished, itis moved up and the next layer is started to be worked on. CLIP technology balances light and oxygen to eliminate the mechanical steps and layers that are the standard DLP process step and allow the production of commercial quality objects at high speed.

BioPrinting involves the liquid mixture of cells, matrix, and nutrients known as bioinks are placed in a printer cartridge and deposited using the patients' medical scans. When a bioprinted pre-tissue is transferred to an incubator, this cell-based pre-tissue matures into a tissue. Also, a matrix may be printed without cell and then populated with cell, in-vivo or ex-vivo.

3D bioprinting for fabricating biological constructs typically involves dispensing cells onto a biocompatible scaffold using a successive layer-by-layer approach to generate tissue-like three-dimensional structures. Given that every tissue in the body is naturally composed of different cell types, many technologies for printing these cells vary in their ability to ensure stability and viability of the cells during the manufacturing process. Some of the methods that are used for 3D bioprinting of cell some of the printing techniques mentioned above as well as extrusion printing into a support gel

### SUMMARY OF THE INVENTION

Various embodiments of methods for treating cancer in a patent are disclosed. One cancer treatment method comprises the step of introducing a collagen device having a DDR1 peptide into the patient, whereby the DDR1 peptide is delivered to the cancer.

Another cancer treatment method comprises the step of introducing a DDR1 peptide into the patient, whereby the DDR 1 peptide is delivered to the cancer.

Another cancer treatment method comprises the steps of introducing a DDR1 peptide into the patient, whereby the DDR1 peptide is delivered to the cancer; and administering an anti-cancer therapeutic agent to the patient.

Another cancer treatment method comprises the steps of introducing a collagen device having a DDR1 peptide into the patient, whereby the DDR1 peptide is delivered to the cancer; and administering an anti-cancer therapeutic agent to the patient.

Another cancer treatment method comprises administration of a therapeutic agent derivatized with a targeting peptide that binds receptors present on cancer cells.

Another cancer treatment method comprises administration of a therapeutic agent derivatized with a synthetic receptor or targeting peptide that binds to a collagen device, including DDR receptors, select integrin receptors (α1β1, α2β1, α10β1 and α11β1), and select immunoglobulin (IgG)-like receptors, glycoprotein VI (GPVI), leukocyte-associated immunoglobulin-like receptor 1 (LAIR-1), and osteoclast-associated receptor (OSCAR), and introducing the collagen device into the patient, whereby the therapeutic agent is delivered to the cancer.

Various embodiments of collagen devices and methods of forming same are disclosed as well.

Further aspects of the invention include the following:
1. A method for treating cancer in a patient, comprising the steps of: introducing a collagen device including a DDR1 peptide into the patient, whereby the DDR1 peptide is delivered to the cancer.
2. The method of aspect 1, where the collagen device is an injectable device comprising any of type III collagen, rh type III collagen (modified with DDR1 binding sites or native) including a DDR1 peptide.
3. The method of aspect 1, where the collagen device is an onlay device or film comprising any type III collagen or rh type III collagen (modified with DDR 1 binding sites or native) including a DDR1 peptide.
4. A method for treating cancer in a patient, comprising the steps of: introducing a DDR1 peptide into the patient, whereby the DDR1 peptide is delivered to the cancer.
5. The methods of aspects 1 or 4, where the DDR1 peptide or collagen device including a DDR1 peptide is dispersed in a degradable carrier.
6. The method of aspect 5, where the carrier is selected from gelatin or other collagens.
7. The method of aspect 5, where the carrier is selected from synthetic degradable polymers.
8. The device of aspect 7, where the carrier is selected from PEG, PLA, PGA, PLGA, polymers thereof, and the like.
9. The aspects of claim 1 or 4, where the DDR1 peptide dispersed in a carrier or collagen device including a DDR 1 peptide dispersed in a carrier is injectable.
10. A method for treating cancer in a patient, comprising the steps of: introducing a collagen device including a DDR1 peptide; and into the patient, whereby the DDR1 peptide is delivered to the cancer; and co-administering an anti-cancer therapeutic agent.
11. The method of aspect 10, where the therapeutic agent is a cancer API.
12. The method of aspect 10, where the therapeutic agent is a cancer vaccine.
13. The method of aspect 10, where therapeutic agent is an inhibitor/degrader of HPK1.
14. A peptide-drug conjugate comprising [(therapeutic agent)-(linking peptide-(DDR1 peptide)]m as described in the detailed description of the invention) and referred to herein as CONSTRUCT 1.
15. A peptide-drug conjugate comprising [(therapeutic agent)-(linking peptide)-(non-DDR1 peptide)], as described in the detailed description of the invention) and referred to herein as CONSTRUCT 2.
16. A peptide-drug or collagen III receptor-drug conjugate comprising [(therapeutic agent)-(linking peptide)-(collagen III receptor or targeting peptide)], as described in the detailed description of the invention) and referred to herein as CONSTRUCT 3.
17. A collagen I receptor-drug conjugate comprising [(therapeutic agent)-(linking peptide)-(collagen I receptor)], as described in the detailed description of the invention) and referred to herein as CONSTRUCT 4.
18. A synthetic cellular receptor-drug conjugate comprising [(therapeutic agent)-(linking peptide)-(synthetic cellular receptor)], as described in the detailed description of the invention) and referred to herein as CONSTRUCT 5.
19. A peptide-drug conjugate comprising [(therapeutic agent)-(linking peptide)-GPRGQOGVMGFO) (SEQ ID NO: 3)], as described in the detailed description of the invention) and referred to herein as CONSTRUCT 6.
20. A method for treating cancer in a patient, comprising the steps of: administering conjugate CONSTRUCT 1 into the patient, whereby the conjugate is delivered to the cancer.
21. A method for treating cancer in a patient, comprising the steps of: administering conjugate CONSTRUCT 2 into the patient, whereby the conjugate is delivered to the cancer.
22. A method for treating cancer in a patient, comprising the steps of: binding conjugate CONSTRUCT 3 to a collagen device; and introducing the collagen device into the patient, whereby the conjugate is delivered to the cancer.
23. A method for treating cancer in a patient, comprising the steps of: administering conjugate CONSTRUCT 4 into the patient, whereby the conjugate is delivered to the cancer.
24. A method for treating cancer in a patient, comprising the steps of: binding conjugate CONSTRUCT 4 to a collagen I device; and introducing the collagen device into the patient, whereby the conjugate is delivered to the cancer.
25. A method for treating cancer in a patient, comprising the steps of: binding conjugate CONSTRUCT 5 to a collagen device; and introducing the collagen device into the patient, whereby the conjugate is delivered to the cancer.
26. A method for treating cancer in a patient, comprising the steps of: administering conjugate CONSTRUCT 6 into the patient, whereby the conjugate is delivered to the cancer.
27. A method for treating cancer in a patient, comprising the steps of: binding conjugate CONSTRUCT 6 to a collagen device; and introducing the collagen device into the patient, whereby the conjugate is delivered to the cancer.
28. The method of aspect 22, where the collagen receptor is selected from DDR1, DDR2, GPVI, LAIR-1, and OSCAR receptors.
29. The method of aspect 22, where the collagen binding peptide is a sequence from vWF.
30. The method of aspect 29, where the collagen binding peptide is selected from:
   a. Any peptide containing the sequence GxRGQOGVMGFO (SEQ ID NO: 1).
   b. Any peptide containing the sequence RGQOGVMGF (SEQ ID NO: 2).
   c. Any peptide containing the sequence GPRGQOGVMGFO (SEQ ID NO: 3).
31. The device of aspect 2, in which CONSTRUCT 1 is dispersed.
32. The device of aspect 31, where CONSTRUCT 1 is dispersed in a degradable carrier.
33. The device of aspect 32, where the carrier is selected from gelatin or other collagens.
34. The device of aspect 32, where the carrier is selected from synthetic degradable polymers.
35. The device of aspect 34, where the carrier is selected from PEG, PLA, PGA, PLGA, polymers thereof, and the like.
36. The device of aspect 3, in which CONSTRUCT 1 is dispersed.
37. The device of aspect 36, where CONSTRUCT 1 is dispersed in a degradable carrier.
38. The device of aspect 37, where the carrier is selected from gelatin or other collagens.
39. The device of aspect 37, where the carrier is selected from synthetic degradable polymers.
40. The device of aspect 39, where the carrier is selected from PEG, PLA, PGA, PLGA, polymers thereof, and the like.
41. The device of aspect 22, in which CONSTRUCT 1 is dispersed.
42. The device of aspect 41, where CONSTRUCT 1 is dispersed in a degradable carrier.
43. The device of aspect 42, where the carrier is selected from gelatin or other collagens.
44. The device of aspect 42, where the carrier is selected from synthetic degradable polymers.
45. The device of aspect 44, where the carrier is selected from PEG, PLA, PGA, PLGA, polymers thereof, and the like.
46. The device of aspect 24, where CONSTRUCT 4 is dispersed in a degradable carrier.
47. The device of aspect 46, where the carrier is selected from gelatin or other collagens.
48. The device of aspect 46, where the carrier is selected from synthetic degradable polymers.
49. The device of aspect 48, where the carrier is selected from PEG, PLA, PGA, PLGA, polymers thereof, and the like.
50. The device of aspect 22, where the synthetic cellular receptor is selected from DDR1, DDR2, GPVI, LAIR-1, and OSCAR receptors.
51. The device of aspect 22, to which the CONSTRUCT 3 is bound to the type III collagen through the synthetic cellular receptor of aspect 50.
52. The device of aspect 22, to which the CONSTRUCT 3 is bound to the type III collagen through collagen binding peptide(s) present in vWF.
53. The device of aspect 52, where the collagen binding peptide is selected from:
   d. Any peptide containing the sequence GxRGQOGVMGFO (SEQ ID NO: 1).
   e. Any peptide containing the sequence RGQOGVMGF (SEQ ID NO: 2).
   f. Any peptide containing the sequence GPRGQOGVMGFO (SEQ ID NO: 3).

### BRIEF DESCRIPTION OF THE DRAWINGS

Embodiments of the invention are further described but are in no way limited by the following drawings.

FIG. 1 is a list of amino acid functional groups frequently targeted for conjugation (SEQ ID NO: 73);

FIG. 2 is a list of primary amine reactive chemical groups;

FIG. 3 is the reaction scheme for NHS ester-mediated coupling of carboxylic acid to a primary amine of a biomolecule;

FIG. 4 is the reaction scheme for imidoester coupling to a primary amine of a biomolecule;

FIG. 5 is the reaction scheme for carbodiimide-mediated coupling of carboxylic acid to a primary amine of a biomolecule;

FIG. 6 is the reaction scheme for carbodiimide-mediated coupling of carboxylic acid to a primary amine of a biomolecule in conjunction with sulfo-NHS;

FIG. 7 is the reaction scheme for maleimide coupling to a sulfhydryl of a biomolecule;

FIG. 8 is the reaction scheme for iodoacetyl coupling to a sulfhydryl of a biomolecule;

FIG. 9 is the reaction scheme for pyridyl disulfide coupling to a sulfhydryl of a biomolecule;

FIG. 10A shows the aryl ketone coupling mechanism;

FIG. 10B is the reaction scheme for aryl ketone coupling to a substrate having abstractable hydrogen; and

FIG. 11 shows the chemical structure of the reagent tetrakis (4-benzoylbenzyl ether) of pentaerythritol (TBBE), which includes four latent reactive benzophenone groups.

### DETAILED DESCRIPTION OF THE INVENTION

Provided herein are collagen devices for treating cancer, methods for forming such collagen devices, and methods of using such collagen devices in the treatment of cancer.

An aspect of the invention pertains to the utilization of 3D printing to digitally process dental and medical devices as well as ECM scaffolds and cellular scaffolds primarily composed of collagen, modified collagen and/or collagen-based peptides. In an exemplary embodiment, to produce polymerizable peptides, collagen is digested with an enzyme, then the peptides are modified with functional groups that can be polymerized with radiation. These modified peptides are then formulated with initiator(s), crosslinker(s), solvents and/or other additives to create the desired design inputs for a particular dental or medical application. This formulation can then be 3D printed.

In another exemplary embodiment, collagen is digested to create peptides, and other peptides are added or subtracted to generate customized desired design inputs for a particular dental or medical application (see, e.g., Applicant's International Patent Application No. PCT/US2021/049646, published as WO 2022/060622 A1, the disclosure of which is incorporated herein by reference). These newly formulated peptides are then modified with functional groups that can be polymerized with radiation. These modified peptides are then formulated with initiator(s), crosslinker(s), solvents and/or other additives to create the desired design inputs for a particular dental or medical application. This formulation then can be 3D printed. Another example involves extruding collagen that has been modified so that it is soluble in a solvent and optionally modified with functional chemistry so that an energy driven, post-process can be carried out. In exemplary embodiments, the extrudable collagen can also contain other collagen-based peptides, such as collagen mimetic peptides (“CMPs”, also known as collagen-hybridizing peptides (“CHPs”), to amplify collagen biological processes. In exemplary embodiments, the extrudable collagen can also contain other bioactive based peptides and growth factors, and cytokines to enhance the healing process.

Provided below are exemplary procedures for producing collagen-based dispersions or reconstituted collagen matrix that are molded. An acid dispersion of collagen fibers with a solid content of about 0.1 to 1.5% (w/w) is first prepared. Both inorganic and organic acids or bases can be used. For example, a 0.05 M to 0.1 M lactic acid dispersion of collagen that has a pH about 2.3 to 2.5 is prepared. An aliquot of the insoluble collagen fibers is weighed and dispersed in the acid solution, homogenized using a commercial homogenizer, and filtered with a stainless-steel mesh filter to obtain a collagen dispersion. The collagen dispersion prepared is then placed in a flask and reconstituted by adjusting the pH to the isoelectric point of collagen (i.e., pH 5.0), by adding NH4OH. The reconstituted collagen fibers are then removed from the beaker and placed on a stainless-steel screen to remove excess solution until the desired solid content of the reconstituted collagen fibers was reached.

In another exemplary embodiment, making a collagen dispersion and reconstituted collagen fibers involves the use of bases. Similar to making the acidic dispersion, base dispersion of collagen fibers of about 0.2-2.0% (w/w) is dispersed in 0.001M-0.05M NaOH, homogenized, filtered to form the final collagen dispersion, and de-gassed under vacuum. The dispersion is then neutralized by adding HCI. The neutralized collagen dispersion is centrifuged and decanted to a certain amount of supernatant to reach the desired density of the final reconstituted collagen mixture. The mixture can then be crosslinked and placed in molds and may be freeze dried and finally sterilized.

The composition of the tumor extracellular matrix (ECM) is beginning to gain recognition as playing a critical role in the tumor microenvironment. During tumor growth, the ECM surrounding - the tumor undergoes dramatic remodeling. The normal matrix is degraded and substituted with an ECM that has a higher collagen density and increased stiffness. The structure and density of this tumor-specific ECM supports tumor growth and metastasis, and is therefore associated with poor prognosis in several types of cancer, such as breast, pancreatic, and gastric cancers. This dense tumor ECM acts as a physical barrier, blocking the infiltration of immune cells, such as T cells and macrophages, which can attack and kill cancerous cells. While collagen in healthy tissue acts as a network for the migration of activated T cells, dense collagen in the tumor microenvironment hinders immune cell infiltration and T cell migration (Boissonnas 2007). Also, in addition to acting as a physical barrier, a collagen-dense ECM reduces the proliferation of cytotoxic T cells, a subset of T cells that can directly kill malignant cells (Kuczek 2019), and directs tumor-associated macrophages to acquire an M2-like phenotype that promotes tumor growth through immunosuppression and angiogenesis (Liu 2021). The dense ECM can also act as a reservoir for immunomodulatory growth factors, and as a barrier that hinders chemotherapy diffusion.

Di Martino et al. (2021) demonstrated that cancer cells can secrete type III collagen into their extracellular space, inducing the cells into a dormant state. This is driven by activation of the discoidin domain receptor tyrosine kinase (DDR1), a receptor that is overexpressed in certain tumors and recognizes specific, conserved sequences within collagen. The ECM around these dormant cells is characterized by a loose, wavy collagen matrix enriched in part by DDR1-induced type III collagen that allows immune cell invasion, such as T cells, and chemotherapy diffusion to the tumor cells. Upon dormant cell awakening, the extracellular domain of DDR1 is cleaved by MMPs, and this domain of DDR1 is responsible for collagen fiber alignment that contributes to the highly aligned and dense ECM, ultimately preventing T cell invasion and contributing to the failure of anti-tumor immunity. Monoclonal antibodies that recognize the extracellular domain of DDR1 can capture and prevent it from participating in collagen fiber alignment, allowing immune cell invasion (Sun 2021). Similarly, collagen-based peptides that contain the conserved consensus sequence recognized by the DDR1 receptor, said peptides referred to herein as DDR1 peptides or DDR1 binding peptides, could capture the extracellular domain of DDR1 and prevent ECM reorganization. Alternately, these peptides could also act as an agonist for membrane-bound DDR1 to induce expression of the loose, wavy type III collagen that makes up the ECM associated with tumor dormancy. T cells could also be genetically engineered or induced to express DDR1 peptide, promoting their migration in the collagen matrix and tumor infiltration.

Recombinant human (rh) collagen is increasingly being used in medical devices and tissue engineering scaffolds due to concerns over the ability of animal-derived collagen to evoke an immune response in a small percentage of the population. Recombinant human collagen has been developed in several stable or transient expression systems, including yeast, mammalian cells, bacterial systems, and tobacco plants, as an alternative source of collagen with modifications that mimic the biological and mechanical functions of native collagen. This is especially useful when the specific collagen to be isolated, such as type III collagen, is either low in abundance, difficult to purify from donor tissue, or difficult to separate from other collagen types. This technology can also be utilized to produce recombinant polypeptides based on human collagen, with sequence modifications aimed at increasing the density of cell binding sites or increasing the homogeneity of collagen fragments in the scaffolds, with desired specifications such as increased DDR1 binding sites.

DDR1 has been shown to regulate collagen fiber alignment and induce type III collagen expression, ultimately controlling tumor cell dormancy. Various embodiments of engineered collagen devices that could regulate tumor cell dormancy are discussed below. In exemplary embodiments, such devices can be introduced to a patient at or near a tumor site locally by various methods, e.g., devices can be surgically implanted, injected or placed cutaneously (i.e., topically/transdermally), or placed non-surgically such as through a needle or catheter in various embodiments.

Film: A film of type III collagen film can be utilized in various exemplary embodiments. The film should be optimized for degradation over a period of time by controlling crosslinking, density and porosity. The number of DDR1 binding sites in these devices can be increased by synthesizing DDR1 binding peptides and crosslinking the peptides into the device. As the device degrades, both the native DDR 1 binding peptides and the added DDR binding peptide release over time. The number of DDR 1 binding sites (peptides) in these devices can also be increased by taking isolated DDR 1 peptides from digested type III collagen and crosslinking the peptides into the device. As the device degrades, the native DDR 1 binding peptides as well as the added peptides release over time. The DDR1 binding peptides can be added to the device (not crosslinked to the device) so that they can release over time. One can decrease the level of DDR1 peptide in the device by blending the device with a variety of collagens, ECMs, GAGs and other biocompatible polymers. One can also vary the DDR1 peptide in type III collagen by producing rh type III collagen with varying amounts of DDR1 binding peptides through genetic engineering.

As mentioned previously, DDR1 receptor binds to and is activated by specific, evolutionarily conserved sequences within collagen. The sequence GVMGFO (SEQ ID NO: 5), where the amino acid O is hydroxyproline, has been identified as a DDR1 binding motif in collagen. CHPs and CMPs are synthetic peptide sequences with repeating units of Gly-Xaa-Yaa amino acid triplets that mimic the hallmark sequence of collagen. These peptides can be designed and synthesized to contain cell interacting domains and collagen receptor binding motifs, such as the DDR1 binding motif GVMGFO (SEQ ID NO: 5). These sequences are typically flanked by repeats of GPO or GfO, where f is 2S,4S fluoroproline, to maintain triple helical hybridization propensity. Therefore, these peptides may be in a single, double, or triple helical form. Other binding sites and sequences that can bind to and activate DDR1, as determined by collagen binding assays, cell membrane-bound DDR1 autophosphorylation, and activation of the downstream signaling pathway, could also be incorporated. In various exemplary embodiments, these peptides could also be modified with positive or negatively charged amino acid side chains to control their diffusion through an electric field. Listed below are exemplary embodiments of potential DDR1-activating or DDR1-binding peptide sequences that can bind to and/or activate DDR1 on their own, or as mentioned previously, can also be added or crosslinked to a device.
a. Any peptide containing the sequence GVMGFO (SEQ ID NO: 5) or GVMGFP (SEQ ID NO: 6).
b. Any peptide containing the sequence GPSGFO (SEQ ID NO: 7) or GPSGFP (SEQ ID NO: 8).
c. Any peptide containing the sequence GPRGFO (SEQ ID NO: 9) or GPRGFP (SEQ ID NO: 10).
d. Any peptide containing the sequence GARGFO (SEQ ID NO: 11) or GARGFP (SEQ ID NO: 12).
e. Any peptide containing the sequence GQOGFO (SEQ ID NO: 13) or GQOGFP (SEQ ID NO: 14).
f. Any peptide containing the sequence GAMGFO (SEQ ID NO: 15) or GAMGFP (SEQ ID NO: 16).
g. Any peptide containing the sequences in items 1-6 flanked by any number of GPO or GfO amino acid triplets, such as GPO(n)-GVMGFO-GPO(n) (SEQ ID NO: 43) and GfO(n)-GVMGFO-GfO (n) (SEQ ID NO: 44), where O is the amino acid hydroxyproline, f is 2S,4S fluoroproline, and n is the number of GPO or GfO repeats.
h. Any peptide sequence that can bind to DDR1, induce autophosphorylation of cell membrane bound DDR1, or can activate the DDR 1 signaling pathway.
i. Any peptides from items 1-8 in single, double, or triple helical form.
j. Any peptides from items 1-9 that have been modified with positive or negatively charged amino acid side chains.

Layered films of treating cancer: The type III collagen that is produced as a result of DDR1 activation contributes to an extracellular matrix that is loose and allows immune cell invasion and chemotherapy diffusion, ultimately keeping tumor cells dormant and quiescent. Discussed below are various embodiments of engineered type III collagen devices that can be surgically implanted. One such device contains multiple (e.g., at least two) layers of type III collagen, at least some of which contain DDR1 binding peptide sequence. The first layer is a crosslinked type III collagen such that the crosslinking density results in the layer that will resorb/breakdown via digestion in approximately 6-12 months. The second layer is a crosslinked type III collagen such that the crosslinking density results in the layer that will resorb/breakdown via digestion in approximately 3-6 months. The third layer is a crosslinked type III collagen such that the crosslinking density results in the layer that will resorb/breakdown via digestion in approximately 0-3 months. Some layers may have more, less, or no crosslinking and still breakdown during these timepoints due to dimension (thickness) and density. As this layered device is breaking down over the 12-month period, it will release the DDR 1 binding peptide from the collagen so that it can bind to the cleaved DDR I receptor to prevent endogenous collagen fiber alignment and/or induce endogenous type III collagen production. The concentration of the DDR1 peptides can be increased in each layer by chemically binding the DDR1 binding peptide into the device. As the device is digested, increasing amounts of DDR1 binding peptide will be released. The concentration of the DDR1 binding peptide can be decreased in each layer by blending the type III collagen with other biomolecules or biocompatible polymers. As the devices is digested/degrades, a lower amount of DDR1 binding peptide will be released. The DDR 1 binding peptide can be added to the device and not bound so that it releases over time. In particular, a burst effect of the DDR1 binding peptide upon placement of the device would be advantageous to immediately arrest the cancer. Of course, a cancer API can be added to the device for local treatment of the cancer. Also, a modified DDR1 binding peptide bound to an API can be added to the device. In this approach, the API modified DDRI binding peptide would release from the device and selectively bind to the cancer cell, thereby delivering the API directly to the cancer cell. Of course, the device can be engineered to be one or more than 3 layers and perform similarly by releasing DDR 1 binding peptide over extended periods of time. Instead of utilizing type III collagen as a carrier, biodegradable polymers, of varying degradation rates, loaded with the DDR1 binding peptide at varying concentration can result in a device that can arrest cancer over an extended period of time. In various exemplary embodiments, the device can also be engineered to release for more than 12 months. In other embodiments, the device can have fewer or more than three layers. In various exemplary embodiments, these devices can be manufactured via additive manufacturing or molding.

Biodegradable films containing the DDR 1 peptide and type III collagen: In various exemplary embodiments, I above-described films can also be engineered by utilizing biodegradable biomolecule or polymers as a carrier for a DDR1 peptides and collagen type III. For example, the DDR1 peptide can be added to 3 layers where each layer is a different ratio of polylactic acid (PLA) and polyglycolic acid (PGA), resulting in different release rates of the DDR1 peptide and collagen type III. In another example, 3 layers of hyaluronic acid containing the DDR1 peptide or collagen type III where the HA has different cross-linked density resulting in each layer degrading and releasing the DDR1 peptide at 3 different rates. In various exemplary embodiments, DDR1 peptides or type III collagen can also be added to other collagen types resulting in various release profiles, ultimately releasing the DDR1 binding peptide.

Injectable Devices for treating cancer: In various exemplary embodiments, the device may also be an injectable device containing the DDR1 peptide. In various embodiments, the injectable device may comprise type III collagen, rh type III collagen (modified with DDR1 binding sites or native), DDR1 peptide, and/or carriers and the like. These DDR1 binding peptides, whether bound to a collagen matrix or released, could capture the extracellular domain of DDR 1 and prevent it from participating in ECM reorganization. As previously mentioned, the peptides could also activate membrane-bound DDR1 to induce type III collagen expression and production, contributing to the ECM associated with tumor dormancy. The DDR 1 peptide that binds to cancer cells can also have an API bound to the peptide as a means of delivering the API selectively to the cancer cell. Another embodiment includes collecting T cells and genetically engineering or inducing them to express DDR1 peptide to promote their migration into the collagen matrix and tumor infiltration.

The overall approach to an injectable device is to provide the DDR1 peptide to the cancer over an extended period of time. There are many approaches to engineer this device, including the following exemplary techniques:
1) Type III collagen particles can be formed at various sizes, densities and crosslinking concentrations such that the particles break down at different rates thereby releasing the naturally present DDR1 peptide. For example, large, highly dense and crosslinked type III collagen particles will degrade slower than small, less dense and lower crosslinked/non-crosslinked particles. While the type III collagen particles are supplying the DDR1 peptide to the cancer cells, the particles can also act as a carrier as well. The particles can be loaded with additional DDR1 binding peptide to increase the overall DDR1 peptide concentration.
2) Many other carriers, including particle form, can be utilized to deliver the DDR1 peptide over extended period of times to the cancer cells: type III collagen, other collagen types, glycosaminoglycans (GAGs), PLA, PGA, poly lactic-co-glycolic acid copolymer (PLGA), polyurethane (PU), and other biodegradable/bioresorbable polymers, hydrogels and biomolecules.
3) The particles described above can be suspended in a variety of biocompatible aqueous solutions including water, buffers, saline that may include other osmotic agents. The suspension of particles, for most cases, would occur just before injection. Of course, the aqueous suspension solution can also contain the DDR1 peptide, which also includes digested type III collagen fragments.
4) The above particles utilized in the injectable device could also be added to the films described above.
5) Much like the DDR1 peptide bound to an API utilized in the film device, that molecule/peptide can be utilized in the injectable device.
6) In the above injectable examples, genetically engineered collagen with added DDR1 peptides can be utilized.

Onlay devices for treating cancer: In various exemplary embodiment, the devices that are described in the above sections on Injectable Devices and Films for cancer treatment can also be used topically (i.e., cutaneously/transdermally) to treat cancer. Other topical devices are possible for delivering the DDR 1 peptides to the cancer site. For example, type III collagen can be processed into a soluble gelatin form and applied topically. The type III gelatin can also contain additional DDR1 peptide to vary concentration. The DDR 1 peptide can be added to numerous topical carrier formulations to deliver the peptide to the treatment site. Many of these formulations are well known in the art and include, for example, micelles, lipid nanoparticles, lipid carriers, microemulsions, and liposomes. In another embodiment, the DDR1 peptide bound to an API described in the film device, that molecule/peptide can be utilized for the topical device.

Other methods to vary the DDR1 peptide concentration in collagen/biomolecules: The type III collagen contains the DDR1 peptide that binds to the DDR1 binding site. In an exemplary embodiment, a rh type III collagen can be engineered to containing increased amount of DDR1 binding sites. This approach is another way to control the concentration of DDR binding peptides in the devices described above. In fact, the DDR1 binding site could be incorporated in any protein or any type of collagen using this technology. The DDR1 peptide itself can also be produced in this manner. Many proteins or biomolecules can be genetically engineered to include a DDR1 peptide sequence. The new proteins or biomolecules can be designed similarly to what is disclosed in this application.

In various exemplary embodiments, cancer-killing T cells could be genetically engineered or induced to express DDR1 peptide, promoting their migration in the collagen matrix and tumor infiltration.

The modified cells can be incorporated back into the patient to treat the cancer. It is preferred to treat the patient with one of the devices in the disclosure before utilizing the CART-T approach.

Liver and pancreatic environments contain a high enzyme environment. This could result in a rapid degradation of protein-based devices. For the treatment of these environments, the films disclosed above could contain a layer of biodegradable polymer (through a hydrolysis process) to help act as a scaffold or possible DDR1 peptide depot. The above biodegradable polymers can also be utilized in this approach.

While using type III collagen has been described herein as a way to deliver the DDR1 peptide to the cancer in a patient, in other exemplary embodiments. collagen can also act a scaffold for tissue regeneration. Many of the devices described herein also function as tissue regenerative devices.

While type III collagen contains the DDR1 binding site, one can genetically engineer most protein or biomolecule to include the DDR1 binding site and design similar devices above utilizing these novel biomolecules. While type III collagen has a typical DDR1 binding motif that can activate DDR1, other types of collagen, such as type II collagen and type IV collagen, also have this DDR1-activating DDR1 binding motif, or can be engineered to have this binding site. Other types of collagen may have sequences that have a weaker affinity for DDR1 and can also activate DDR1. As such, they can also be used in the devices mentioned above.

DDR peptides to enhance the immune response to co-administered therapeutic agents to treat cancer: The innate immune system routinely eradicates cancerous cells (Pandya et al. 2016), but sometimes such cells evade immune cells and form tumors. As Gonzalez et al. (2018) assert, “In principle, tumor development can be controlled by cytotoxic innate and adaptive immune cells; however, as the tumor develops from neoplastic tissue to clinically detectable tumors, cancer cells evolve different mechanisms that mimic peripheral immune tolerance in order to avoid tumoricidal attack.”

While treating cancer in a patient by delivering a DDR1 peptide, or with an implanted collagen device to deliver a DDR1 peptide is novel, unrelated drug-based and emerging cancer therapies are known and practiced routinely. These include cancer APIs intended to kill or inhibit metabolism and proliferation of tumor cells, such as bleomycin, capecitabine, and dacarbazine, doxorubicin, eribulin, gemcitabine, ixabepilone, lapatinib paclitaxel, and vinblastine sulfate, (Cortazar et al. 2012) as well as newer therapeutics such as cancer vaccines intended to elicit an immune response against tumor-specific antigens and to prevent tumor cells from inactivating the T-cells that destroy them (www.cancer.gov/about-cancer/treatment/drugs/abvd). Additionally, new therapeutic agents are discovered every year. For example, U.S. Pat. Pub. No. 20230022524 (incorporated by reference herein in its entirety) discloses heterobifunctional compounds as inhibitors and degraders of Hematopoietic Progenitor Kinase 1 (HPK1) (You et al. 2021; Si et al. 2020), a negative regulator of T cells (Sawasdikosol et al. 2020).

As explained by the National Cancer Institute at the National Institutes of Health, “Immune checkpoints are a normal part of the immune system. Their role is to prevent an immune response from being so strong that it destroys healthy cells in the body. Immune checkpoints engage when proteins on the surface of immune cells called T cells recognize and bind to partner proteins on other cells, such as some tumor cells. These proteins are called immune checkpoint proteins. When the checkpoint and partner proteins bind together, they send an “off” signal to the T cells. This can prevent the immune system from destroying the cancer.

Immunotherapy drugs called immune checkpoint inhibitors work by blocking checkpoint proteins from binding with their partner proteins. This prevents the “off” signal from being sent, allowing the T cells to kill cancer cells. One such drug acts against a checkpoint protein called CTLA-4. Other immune checkpoint inhibitors act against a checkpoint protein called PD-1 or its partner protein PD-L1. Some tumors turn down the T cell response by producing lots of PD-L1 (www.cancer.gov/about-cancer/treatment/types/immunotherapy/checkpoint-inhibitors).

The development of therapeutic (as opposed to preventative) vaccines has accelerated over the past two decades. Therapeutic cancer vaccines generally aim to induce an immune response that primes endogenous tumor-reactive T cells against both tumor-specific antigens (TSAs) and tumor-associated antigens (TAAs), and prevents T cell inactivation by cancer cells (Lin et al. 2022). Clinical outcomes with therapeutic cancer vaccines have been mixed, ranging from dismal failure to resounding success.

Examples of immunotherapeutic vaccines described in literature include Glycoprotein 100(gp 100) vaccine (Tahaghoghi-Hajghorbani et al. 2023), Epstein-Barr virus (EBV) target antigen vaccine (Taylor et al. 2014), Synthetic long peptide (SLP) vaccine ISA101 (Ding et al. 2022), E6/E7-plasmid (VGX-3100) and E6/E7/Fms-like tyrosine kinase 3 ligand (Flt3L)-plasmid (GX-188E) vaccine (Kim et al. 2014), E6/E7/IL-2 MVA vector vaccine (Harper et al. 2019), CDX-1401 with resiquimod (TLR7/8) and Hiltonol (poly-ICLC, TLR3) (Dhodapkar et al. 2014) vaccine, and Adenovirus (ChAdOx1)/MVA vaccine targeting MAGE-A3 and NY-ESO-1 (McAuliffe et al. 2021).

Examples of immunotherapeutic vaccines that failed to achieve meaningful clinical efficacy described in literature include Rindopepimut (CDX-110) vaccine (Weller et al. 2017), TLR4-agonist-adjuvant (AS02B) MAGE-A3 protein vaccine (Vansteenkiste et al. 2013), Nelipepimut-S plus GM-CSF vaccine (Mittendorf et al. 2019), and single-epitope HLA-II-restricted 15-mer peptide (AE37)+GM-CSF vaccine (Mittendorf et al. 2016).

Examples of commercially available clinical immunotherapeutic vaccines include Atezolizumab (Tecentriq) (www.tecentriq.com), Avelumab (Bavencio) (www.bavencio.com/hcp), Cemiplimab (Libtayo) (www.libtayo.com), Durvalumab (Imfinzi) (www.imfinzi.com), Enzalutamide (XTANDI) (www.xtandi.com), Ipilimumab (Yervoy) (www.yervoy.com), Nivolumab (Opdivo) (www.opdivo.com), Nivolumab+Relatlimab-rmbw (Opdualag) (www.opdualag.com), Pembrolizumab (Keytruda) (www.keytruda.com), Rituximab (Rituxan) (www.rituxan.com), and Tremelimumab (Imjudo) (www.accessdata.fda.gov/drugsatfda_docs/label/2022/7612891bl.pdf).

Numerous other cancer vaccines based upon shared antigens, anonymous antigens, personalized antigens, autologous tumor lysate, and intratumorally administered oncolytic viruses and bacteria are in currently in development or under evaluation in registered clinical trials. For example, Moderna (Cambridge, MA) discloses three pipeline cancer vaccines, Personalized cancer vaccine (PCV) mRNA-4157, KRAS vaccine mRNA-5671, and Checkpoint Vaccine mRNA-4359 on its website (www.modernatx.com/research/product-pipeline). Similarly, other drug manufacturers report cancer vaccines in development.

Lin et al. (2022) conclude, “data suggest that inducing T cells against self proteins, even those overexpressed in tumors, requires an elevated immune response for greatest efficacy.” In a further aspect of the present invention, concomitant use of DDR1 peptides and any anti-cancer therapeutic agent(s) improves the efficacy of said therapeutic agent(s) by elevating the effective immune response. Engineered type III collagen devices (including films, particles, and other carriers) containing DDR 1 binding peptide delivered or implanted as an adjunct to pharmaceutical or immunological cancer therapy prevents the formation of a dense tumor ECM that acts as a physical barrier, blocking the infiltration of immune cells, such as T cells and macrophages that can attack and kill cancerous cells, while the therapeutic agent either attacks the tumor directly or elicits an immune response against the tumor and promotes T cell attack of the tumor. The combined use of DDR1 peptides and checkpoint inhibitors or DDR1 peptides and other cancer vaccines is especially attractive, as each promotes increased T cell activity and such combinations improve the effective T-cell reactivity with tumor cells. Further, concomitant use of DDR1 peptides may enable failed cancer vaccines, as the combined effect of the DDR 1 peptide and the vaccine may achieve a T-cell activity beyond a threshold required for effective eradication of the tumor.

A preferred example is” the combination of DDRI peptides that promote the formation of type III collagen and inhibit the formation of a physical barrier that prevents immune cell-mediated tumor cell death, and therapeutic agents described in U.S. Pat. Pub. No. 20230022524 (incorporated by reference herein in its entirety) that inhibit and degrade HPK 1 that would otherwise mediate T cell evasion by the tumor. Two embodiments of the present invention are particularly applicable, the first being HPK 1 inhibitors/degraders and concomitant engineered type III collagen film containing a DDR1 binding peptide sequence, flanked by repeats of GFO or GPO, where F is fluoroproline and O is hydroxyproline, i.e., (GPO) 6 (SEQ ID NO: 42) on each side to maintain the peptide in triple helical form at physiological temperature, and the second being concomitant particulate comprising one or more layers of degradable polymer(s) containing the DDR 1 peptide that deliver the peptide at a controlled rate as the polymer degrades.

DDR1 targeting peptides for tumor-localized delivery of therapeutic agents: In various exemplary embodiments of the invention, the same DDR1 peptides that promote type III collagen formation can be appended with anti-cancer therapeutic agents (including cancer APIs intended to kill tumor cells and vaccines intended to elicit an immune response against the tumor) tethered through degradable linkers to form peptide-drug conjugates. Chavda et al. (2022) describe the general concept of using peptides as drug transporters in this way. They define peptide-drug conjugates as composed of 1. a homing peptide or device; 2. A cytotoxic payload; and 3. A linker that works synergistically to deliver cytotoxins to the targeted receptor on cancerous cells. The present invention includes such a construct, with DDR1 peptide(s) as the homing peptide, but alternatively includes immunotherapeutics rather than cytotoxics as payloads. U.S. Pat. Pub. No. 20210338558 (incorporated by reference herein in its entirety), claim 1 describes a similar construct composed of 1. A targeting moiety for binding to native collagen fibers; 2. A skin care agent or cosmeceutical agent payload; and 3. an intermediate release linker bound to the skin care agent or cosmeceutical agent; the inventors recognize the peptide GVMGFO (SEQ ID NO: 5) as a DDR1 targeting moiety.

The appropriate linkers for peptide-drug conjugates depend upon specific peptide/drug combinations. Examples of drug-linker-peptide combinations include Gemcitabine-glutaryl linker-[D-Lys6]-Gonadotropin hormone-releasing hormone (GnRH), Paclitaxel-ester linker-Angiopep-2, Paclitaxel-ester linker-Poliglumex, Thapsigargin-ester linker-Tetrapeptide, Doxorubicin-ester linker-GnRH, Maytansinoid-Disulfide Linker-Bicyclic peptide, Doxorubicin-Amide Linker-Tetrapeptide, ¹¹¹ In-DTPA-Amido Linker-D-phe-1-octreotide, hTNF-Amido Linker-NGR, Paclitaxel-Ester Linker-Poliglumex, CLIP71-Amino Linker-LHRH, Gemcitabine-Amide, ester, cathepsin-B, carbamate Linker-Knotting peptide, DOTA-g-aminobutyric acid Linker-Bombesin 7-14, Methotrexate-Amino Linker-Acetyl-YTAIAWVKAFIRKLRK (SEQ ID NO: 4) (Chavda et al. 2022). U.S. Pat. Pub. No. 20210338558 also discloses various intermediate release linkers; these include a protein or a peptide, a non-protein polymer, such as polyethylene glycol (PEG), a PEG-modified protein, and biotin/avidin, biotin/streptavidin, antigen/antibody, or hapten/antibody linkages. Examples of peptide linkers include TGEKP (SEQ ID NO: 17), TGGGGSGGGGTGEKP (SEQ ID NO: 18), modifications of TGGGGSGGGGTGEKP (SEQ ID NO: 18), LRQKDGGGSERP (SEQ ID NO: 19), LRQKDGERP (SEQ ID NO: 20), GGRGRGRGRQ (SEQ ID NO: 21),

QNKKGGSGDGKKKQHI (SEQ ID NO: 22), TGGERP (SEQ ID NO: 23), ATGEKP (SEQ ID NO: 24), GGGSGGGGEGP (SEQ ID NO: 25), as well as derivatives of those linkers in which amino acid substitutions are made, such as the serine(S) residue between the diglycine or polyglycine runs in QNKKGGSGDGKKKQHI (SEQ ID NO: 22) or GGGSGGGGEGP (SEQ ID NO: 25) replaced with threonine (T), the glutamic acid (E) at position 9 in GGGSGGGGEGP (SEQ ID NO: 25) replaced with aspartic acid (D), and other linkers such as glycine or serine repeats. When both the therapeutic agent and the intermediate release linker are derivatized by peptides, the linkage between the therapeutic agent and the intermediate release linker can then be a peptide (amide) bond formed between these peptides. Additional cleavable linkers containing disulfide groups, which can be cleaved by reduction, linkers containing glycols, which can be cleaved by periodate, linkers containing diazo groups, which can be cleaved by dithionite, linkers containing ester groups, which can be cleaved by hydroxylamine, and linkers containing sulfones, which can be cleaved by bases.

Typically, the linkages between the anti-cancer therapeutic and the intermediate release linker, and between the intermediate release linker and the targeting peptide are covalent linkages involving reactive moieties and cross-linking agents known in the art. Additionally, the linkages can be non-covalent through such interactions as hydrophobic/hydrophilic interactions, hydrogen bonding, ionic interactions, or salt links as is understood in the art.

Peptide linkers are especially attractive, as they can be appended seamlessly to synthesized DDR1 peptides of the present invention using a standard peptide synthesizer. Optionally, peptide linkers can be substituted with polyethylene glycol (pegylated) to instill in them desired physical and chemical properties that aid in delivery of the tethered drug. Once formed, a [(DDR1)-(linking peptide)] construct can be conjugated to a therapeutic agent via non-covalent means as listed above, but more typically by covalent bonding, using wet chemical and photo-chemical techniques known in the art.

In some embodiments, the linking peptide can be modified with a thermal or photo-reactive group or groups capable of covalently linking to the therapeutic agent (or to the DDR 1 peptide if not already appended synthetically). In other embodiments, the therapeutic agent can be modified with a thermal or photo-reactive group or groups capable of covalently linking to the linking peptide (or the [(linking peptide)-(DDR1 peptide)] conjugate.

The linking peptide (or the [(linking peptide)-(DDR1 peptide)] conjugate) can be tethered to the therapeutic agent, with the method selected depending upon the chemical structure of the agent. Crosslinking agents that react with various reactive chemical moieties, including hydroxyl (—OH), primary amine (—NH2), sulfhydryl (—SH), and carboxyl (—COOH) are known in the art. A therapeutic agent can readily be modified with a linking peptide of the current invention by reacting the crosslinker first with the agent, then with the linking peptide, or vice versa. This can be accomplished by various methods, including wet chemical and photochemical methods.

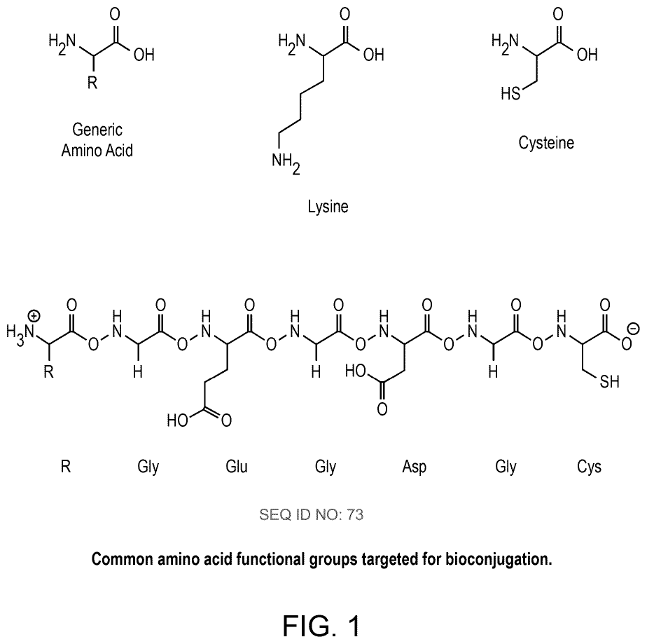

Wet chemical methods useful for the present invention are well described in the Thermo Scientific Crosslinking Technical Handbook (Thermo Fisher Scientific Inc.: Waltham, MA, 2012, available from tools.thermofisher.com/content/sfs/brochures/1602163-Crosslinking-Reagents-Handbook.pdf. Accessed Jul. 28, 2022, incorporated herein by reference). FIG. 1 shows chemical moieties frequently targeted for bioconjugation; Table 1 lists common chemical crosslinkers reactive with such moieties. With respect to the present invention, many anti-cancer vaccines are protein-based vaccines, which are makes them attractive for bioconjugation. In comparison, many APIs, while not protein based, are nonetheless reactive with the same chemical crosslinkers used for bioconjugation.

TABLE 1
Popular crosslinker reactive groups for protein conjugation
(Table 1 of Thermo Scientific Crosslinking Technical Handbook.)

| | Target | |
|---|---|---|
| Reactivity class | functional group | Reactive chemical group |
| Amine-reactive | —NH2 | NHS ester |
| | | Imidoester |
| | | Pentafluorophenyl ester |
| | | Hydroxymethyl phosphine |
| Carboxyl-to-amine | —COOH | Carbodiimide (e.g., EDC) |
| reactive | | |
| Sulfhydryl-reactive | —SH | Maleimide |
| | | Haloacetyl |
| | | (Bromo-or lodo-) |
| | | Pyridyldisulfide |
| | | Thiosulfonate |
| | | Vinylsulfone |
| Aldehyde- | —CHO | Hydrazide |
| reactive i.e., | | Alkoxyamine |
| oxidized | | |
| sugars | | |
| (carbonyls) | | |
| Photo-reactive i.e., | random | Diazirine |
| nonselective, random | | Aryl azide |
| insertion | | |
| Hydroxyl (nonaqueous)- | —OH | Isocyanate |
| reactive | | |
| Azide-reactive | —N3 | Phosphine |

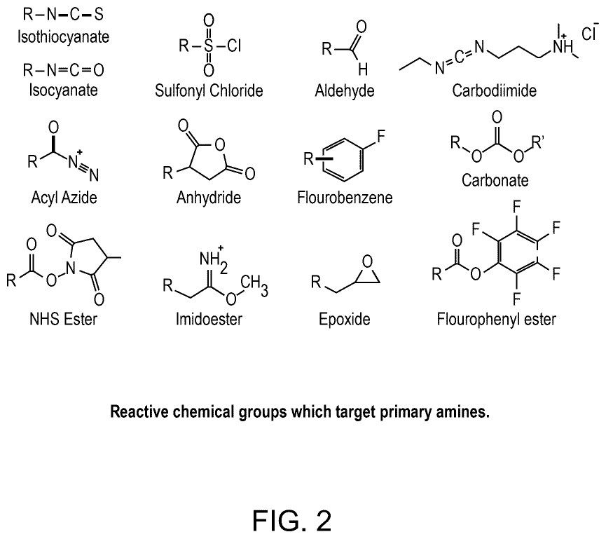

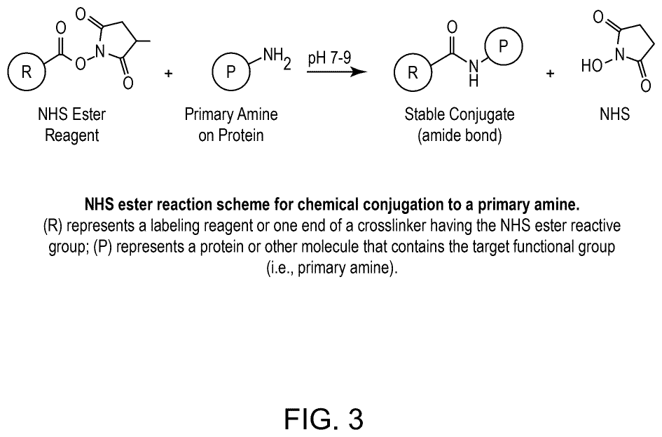

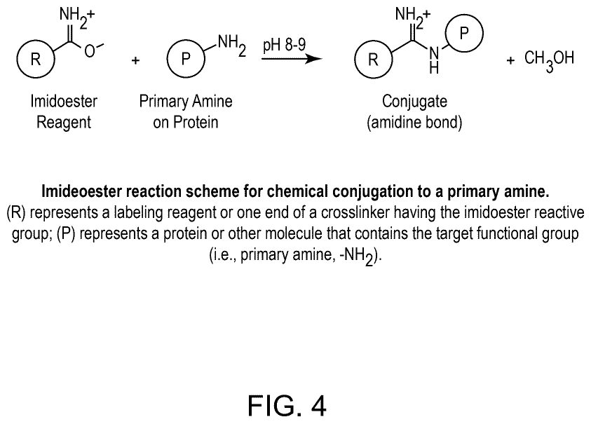

Amine-reactive chemical groups target the primary amines at the N-terminus of the polypeptide chain (alpha-amine), as well as the side chain of lysine residues (epsilon-amine). Due to positive charge at physiologic conditions, primary amines normally locate at the protein surface and thus are accessible for bioconjugation without denaturation. FIG. 2 shows chemical groups that react with primary amines; those most commonly used are NHS esters and imidoesters. FIGS. 3 and 4 show the respective general crosslinking reaction schemes for these reagents.

Carboxylic acid-reactive chemical groups target the carboxyl C-terminus of the polypeptide chain, as well as the side chains of aspartic acid and glutamic acid. Carboxyl groups also normally locate at the protein surface and are thus accessible for bioconjugation.

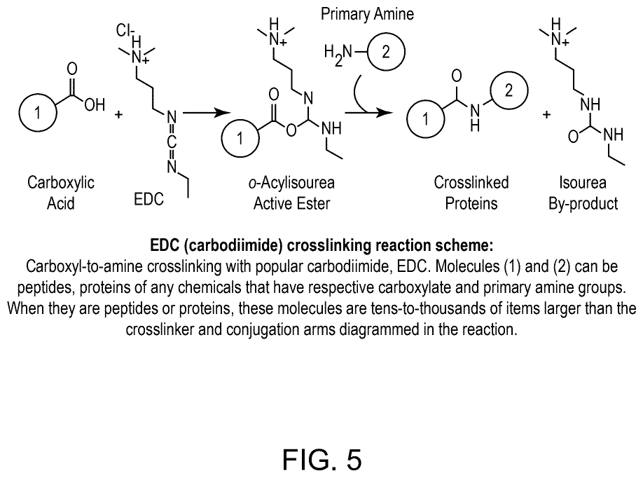

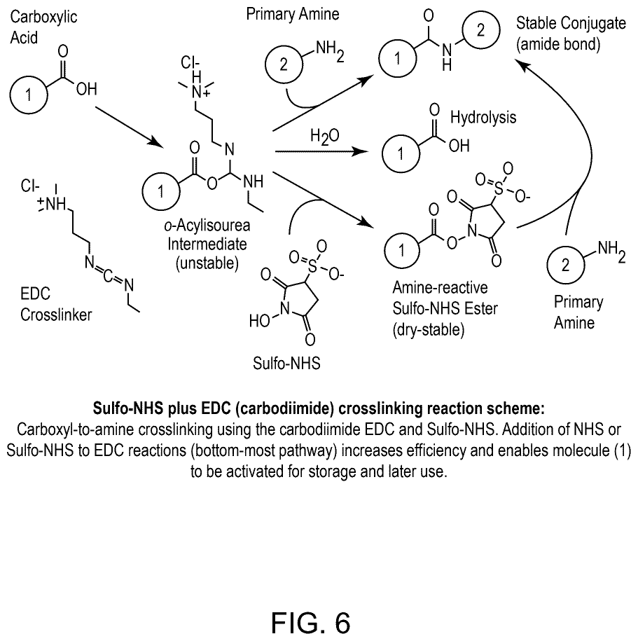

Carbodiimides (EDC and DCC) cause direct conjugation of carboxylates (—COOH) to primary amines (—NH2). FIG. 5 shows the general reaction scheme for EDC-mediated coupling of carboxylic acid of one biomolecule (e.g., linking peptide) to a primary amine of another biomolecule (e.g., the therapeutic agent). FIG. 6 shows the general reaction scheme for EDC coupling in conjunction with sulfo-NHS, which improves reaction efficiency and results in a stable sulfo-NHS derivatized biomolecule (peptide or therapeutic agent) that can be stored and used later reacted with a primary amine of a second complementary biomolecule (therapeutic agent or peptide).

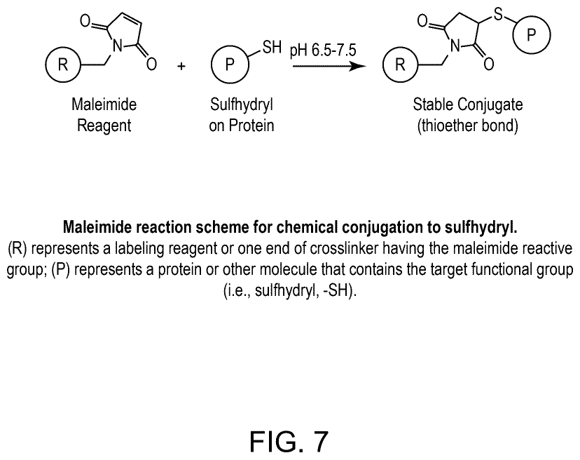

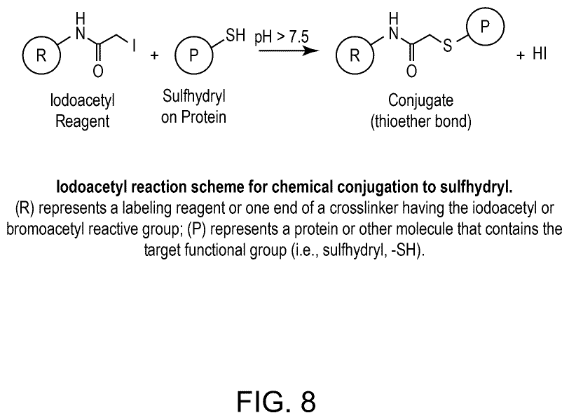

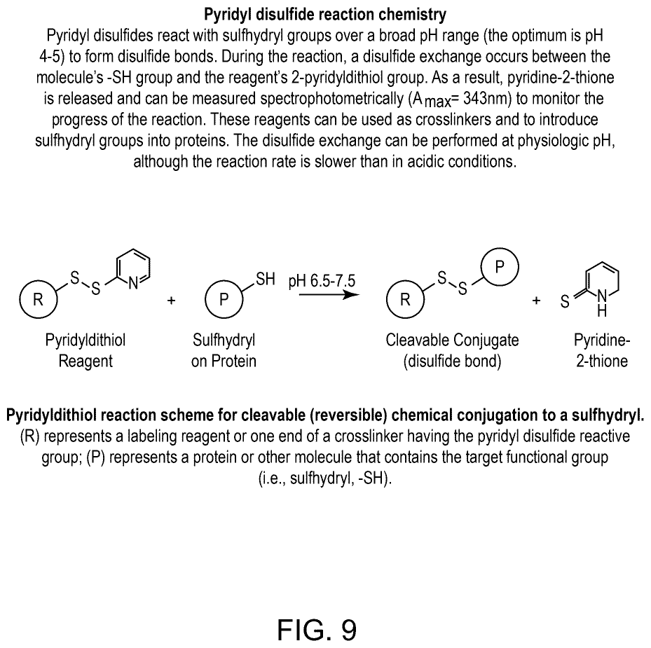

Sulfhydryl-reactive chemical groups target the side chain of cysteine residues. Sometimes disulfide bonds (—S—S—) form between adjacent polypeptide side chains within a protein and must first be reduced to sulfhydryl groups before crosslinking through these groups. Maleimides, haloacetyls and pyridyl disulfide moieties all react with protein sulfhydryl groups. FIGS. 7-9 show the general reaction schemes for maleimide, haloacetyl, and pyridyl disulfide coupling, respectively to a sulfhydryl of a biomolecule. Maleimide chemistry is often used in combination with NHS ester chemistry to produce heterobifunctional crosslinkers that enable controllable, two-step conjugation of peptides to therapeutic agents.

In addition to amino acids bearing amino, carboxyl, or sulfhydryl side groups, other chemicals bearing amino, carboxyl, or sulfhydryl groups can also be used in various exemplary embodiments to end cap the linking peptide. Preferably, such chemicals bear a terminal amino or carboxyl group capable of reacting with the C-terminus carboxyl or N-terminus amino group of the linking peptide, and multiple amino, carboxyl, or sulfhydryl side groups, or combinations thereof, to be targeted for bioconjugation to the therapeutic agent with greater efficiency than the unmodified peptide.

Polyamino-bearing chemicals include propane-1,2,3-triamine (three NH2 groups), tris(2-aminoethyl)amine (three NH2 groups), tetraaminomethane (four NH2 groups), tetra(2-aminoethyl)methane (four NH2 groups), 1,1,2,2-3 ethanetetraamine (four NH2 groups), and 2,2-bis (aminomethyl)propane-1,3-diamine (four NH2 groups).

Polycarboxyl-bearing chemicals include propane-1,2,3-tricarboxylic acid (three COOH groups) and citric acid (three COOH groups).

Polysulfhydryl-bearing chemicals include 2,3-dimercaptopropionic acid (one terminal COOH and one terminal SH group, and one additional SH group) and 2,4-dimercaptopentanedioic (two terminal COOH groups and two SH groups).

Chemicals bearing both polyamino and carboxyl groups include: 2,2-diaminoacetic acid (one terminal COOH and one terminal NH2 group, with one additional NH2 group), 2,4-diaminobutyric acid (one terminal COOH and one terminal NH2 group, with one additional NH2 group), 2,3-diaminopropionic acid (one terminal COOH and one terminal NH2 group, with one additional NH2 group), 2,4-diamino-pentanedioic acid (two terminal COOH groups with two NH2 groups).

In addition to wet chemical methods, photochemical methods are also useful for bioconjugation. U.S. Pat. No. 5,563,056, incorporated herein by reference, describes latent reactive groups and the residue functionality of each upon activation as shown in Table 2.

TABLE 2
Latent Reactive Groups and Their Residues Upon Activation
described in US Patent No. 5,563,056.

| Latent Reactive Group | Residue Functionality |
|---|---|
| aryl azides | amine R—NH—R′ |
| acyl azides | amide R—CO—NH—R′ |
| azidoformates | carbamate R—O—CO—NH—R′ |
| sulfonyl azides | sulfonamide R-SO.sub.2—NH—R′ |
| phosphoryl azides | phosphoramide (RO).sub.2 PO—NH—R′ |
| diazoalkanes | new C—C bond |
| diazoketones | new C—C bond and ketone |
| diazoacetates | new C—C bond and ester |
| beta-keto-alpha- | new C—C bond and beta-ketoester |
| diazoacetates | |
| aliphatic azo | new C—C bond |
| diazirines | new C—C bond |
| ketenes | new C—C bond |
| photoactivated ketones | new C—C bond |
| dialkyl peroxides | ethers |
| diacyl peroxides | esters and new C—C bonds |
| peroxyesters | ethers, esters, and new C—C bonds |

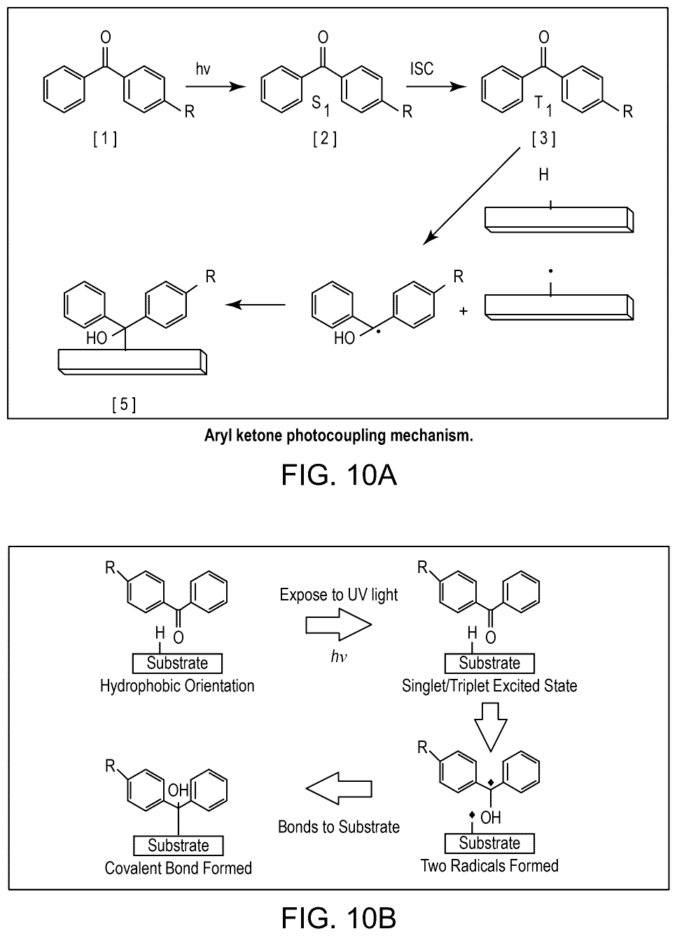

FIGS. 10A and 10B illustrate the general photo coupling reaction of aryl ketones using derivatized benzophenone as example (see Simso EJ. PhotoLink: a new coating concept, Textile Technology International. 1996;1:23, available at p2infohouse.org/ref/33/32073.pdf). U.S. Pat. No. 5,744,515, incorporated herein by reference, describes the associated coupling mechanism as follows. “Photo-reactive aryl ketones such as acetophenone and benzophenone, or their derivatives, are preferred, since these functional groups, typically, are readily capable of undergoing the activation/inactivation/reactivation cycle described herein. Benzophenone is a particularly preferred photo-reactive group, since it is capable of photochemical excitation with the initial formation of an excited singlet state that undergoes intersystem crossing to the triplet state. The excited triplet state can insert into carbon-hydrogen bonds by abstraction of a hydrogen atom (from a support surface, for example), thus creating a radical pair. Subsequent collapse of the radical pair leads to formation of a new carbon-carbon bond. If a reactive bond (e.g., carbon-hydrogen) is not available for bonding, the ultraviolet light induced excitation of the benzophenone group is reversible and the molecule returns to ground state energy level upon removal of the energy source. Photoactivatable aryl ketones such as benzophenone and acetophenone are of particular importance inasmuch as these groups are subject to multiple reactivation in water and hence provide increased coating efficiency. Hence, photo-reactive aryl ketones are particularly preferred.”

Particularly useful in this regard is a heterobifunctional crosslinking agent bearing a photo-reactive benzophenone group at one end and an amine-reactive N-oxy-succinimide (NOS) ester group at the other, the ends tethered together via an epsilon aminocaproic acid (EAC) spacer. Such a compound is useful to bind the NOS end to proteins and peptides through their primary amines, this resulting in a photo-derivatized peptide or protein that can be bioconjugated to any material capable of undergoing the free radical reaction with excited benzophenone. Examples of photo-reactive proteins and peptides are described, respectively, in U.S. Pat. Nos. 5,744,515 and 6,121,027, incorporated herein by reference, including fibronectin, laminin, and type IV collagen, and peptides of fibronectin (RGD, C/H-V, C/H-II), laminin (F-9) and type IV collagen (HEP-III). The chemical methods described in U.S. Pat. No. 6,121,027, incorporated herein by reference, are particularly useful for preparing photo-reactive peptides of the present invention.

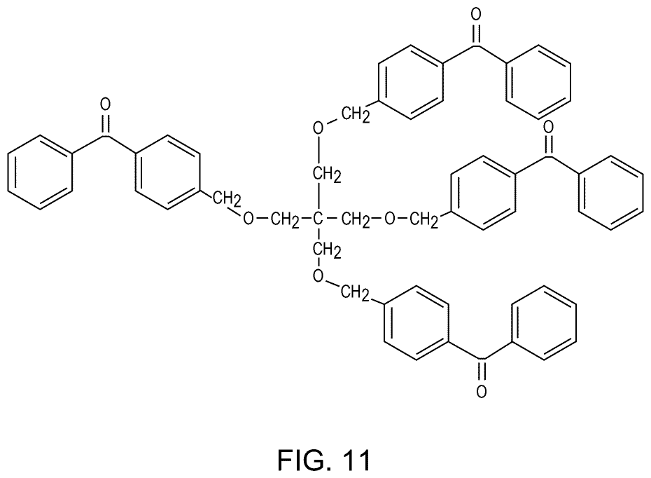

In the case of a photo-derivatized protein-based therapeutic agent, the NOS end of the crosslinking agent is bound to the therapeutic molecule through its primary amines, while the benzophenone end remains free to bind linking peptides of the present invention upon intimate contact and subsequent photo-illumination. In the case of photo-derivatized linking peptides, the benzophenone end of the crosslinking agent remains free to bind the therapeutic molecule at any of the CH groups of the amino acids composing it, with some CH groups more reactive than others. Other photo-crosslinking agents are also useful for binding the linking peptides of the current invention to the therapeutic agents of the current invention. As first disclosed in U.S. Pat. No. 5,414,075, incorporated herein by reference, restrained multifunctional reagents for surface modification containing multiple latent reactive groups are capable of binding first to a support structure upon activation, then subsequently to a different second molecule upon reactivation. For example, FIG. 11 shows the chemical structure of the reagent tetrakis (4-benzoylbenzyl ether) of pentaerythritol (TBBE), which includes four latent reactive benzophenone groups. These multifunctional reagents readily bind to first the therapeutic agents of the present invention, then to the linking peptides of the present invention. In another embodiment, the [(therapeutic agent)-(linking peptide)-(DDR1 peptide)] construct, referred to herein as CONSTRUCT 1 can be formulated with a biodegradable carrier so that it can release over time.

In other embodiments, the [(therapeutic agent)-(linking peptide)-(DDR1 peptide)] construct is added to reconstituted (or otherwise modified) collagen so that it impacts the pharmacokinetics or release characteristics of the former.

Collagen targeting peptides other than DDR1 for tumor-localized delivery of therapeutic agents: In various exemplary embodiments of the present invention, anti-cancer therapeutic agents are delivered to a tumor by exploiting additional ligand-non DDR 1 receptor interactions. This is accomplished using one of two distinct constructs. In the first construct, the afore described [(therapeutic agent)-(linking peptide)] is tethered to a ligand that binds collagen receptors on the tumor cell, including DDR2 receptors, select integrin receptors (α1β1, α2β1, α10β1 and α11β1), and select immunoglobulin (IgG)-like receptors, glycoprotein VI (GPVI), leukocyte-associated immunoglobulin-like receptor 1 (LAIR-1), and osteoclast-associated receptor (OSCAR), and mannose receptors (Agarwal et al. 2019). The selection of ligand depends upon the type of the tumor cell and its collagen receptors. Such ligands are typically peptides, but can be other structures as well. The conjugates are analogous to that of the previous aspect, but with the DDR1 peptide replaced by peptides that bind non-DDR I receptors [(therapeutic agent)-(linking peptide)-(non-DDR 1 peptide)], referred to herein as CONSTRUCT 2. Targeting ligands that bind non-collagen receptors can also be used. In the second construct, the therapeutic agent-linking peptide is tethered to a synthetic receptor or targeting peptide that binds a ligand in collagen III, and the [(therapeutic agent)-(linking peptide)-(collagen III receptor or targeting peptide)] conjugate, referred to herein as CONSTRUCT 3, bound to a collagen III device (particles or film) is placed at or near the tumor site. Alternatively, the therapeutic agent-linking peptide is tethered to a synthetic receptor or targeting peptide that binds a ligand in type I collagen, such as a DDR or integrin receptors to yield a [(therapeutic agent)-(linking peptide)-(collagen I receptor)] construct, referred to herein as CONSTRUCT 4 (particles or film), and delivered to the tumor to exploit the overexpression of type I collagen in many tumors (Kanta 2015; Hsu et al. 2022).

GVMGFO (SEQ ID NO: 5) and some of the additional peptide ligands for DDR1 listed above are also ligands for DDR2, as are additional DDR2 binding peptides in addition to GVMGFO (SEQ ID NO: 5) identified using col-II and col-III toolkit peptides (Agarwal G et al. 2019); some ligands are common to DDR1 and DDR2, while others are specific for DDR1 or DDR2 (Leitinger et al. 2014). DDR2 binding peptides as indicated in Table 3 (Table 1 of Konitsiotis et al. 2008) and Table 4 (Table 1 of Xu et al. 2011) can readily be tethered to a linking peptide in a single synthetic step, and can be appended with the appropriate terminal amino acids for ready chemical coupling to therapeutic agents.

Similar to targeting peptides, in various exemplary embodiments, targeting antibodies can also be used to deliver the therapeutic agent to the tumor site. For example, DDR monoclonal antibodies are described in academic literature (Carafoli F et al. 2013). Like the [(therapeutic agent)-(linking peptide)-(DDR1-peptide)] and [(therapeutic agent-linking peptide-DDR2-peptide)] constructs, a [(therapeutic agent)-(linking peptide)-(DDR monoclonal antibody)] construct also targets DDR receptors.

TABLE 3
DDR2 binding peptides
Guest sequences of peptides used to characterize DDR2-collagen
Interaction¹

| | Peptide | | DDR2 |
|---|---|---|---|
| SEQ ID NO: | Name | Sequence | binding |
| 45 | II-22 | GAOGEDGROGPOGPQGARGQOGVMGFO | + |
| 46 | II-23 | GQOGVMGFOGPKGANGEOGKAGEKGLO | + |
| 47 | III-23 | GPOGPSGPRGQOGVMGFOGPKGNDGAO | + |
| 48 | GPS-GFO | GPSGPRGQOGVMGFO | + |
| 49 | GPS-GVN | GPSGPRGQOGVM | − |
| 50 | GPS-GQO | GPSGPRGQO | − |
| 51 | GFOGPK | GFOGPK | − |
| 52 | GPR-GPK | GPRGQOGVMGFOGPK | − |
| 3 | GPR-GFO | GPRGQOGVMGFO | + |
| 53 | GQO-GFO | GQOGVMGFOGPK | + |
| 5 | GVMGFO | GVMGFOGPK | +/− |
| 54 | GAP-GPR-GFO² | (GAP) [symbol] -GPRGQOGVMGFO-(GAP) [symbol] | +/− |
| 26 | GPOGER | GPOGER | − |
| 55 | GPS-GFO-5A | GPSGARGQOGVMGFO | − |
| 56 | GPS-GFO-6A | GPSGPAGQOGVMGFO | + |
| 57 | GPS-GFO-8A | GPSGPRGAOGVMGFO | +/− |
| 58 | GPS-GFO-9A | GPSGPRGQAGVMGFO | + |
| 59 | GPS-GFO-11A | GPSGPRGQOGAMGFO | + |
| 60 | GPS-GFO-12A | GPSGPRGQOGVAGFO | + |
| 61 | GPS-GFO-14A | GPSGPRGQOGVMGAO | − |
| 62 | GPS-CFO-15A | GPSGPRGQOGVMGFA | +/− |
| 63 | CI-alpha1 | GARGQAGVMGFO | + |
| 64 | CI-alpha2 | GARGEOGNIGFO | − |

¹DDR2 binding denotes binding to His-DDR2 or DS2-Fc. A plus sign (+) indicates a response similar in peptides II-22, III-23, or GPS-GFO (control); a minus sign (−) Indicates less than 20% of control; +/ − indicates 20-80% of control.
GAP-GPR-GFO: non-helical peptide flanked by (GAP) [symbol] instead of (GPP) [symbol] flanking all other peptides. [symbol]
indicates data missing or illegible when filed

TABLE 4
DDR1 and DDR2 binding peptides
Guest sequenecs of peptides used to characterise
DDR collagen interaction

[symbol]

| SEQ ID | Peptide | | DDR1 | DDR2 |
|---|---|---|---|---|
| NO: | Name | Sequence | binding | binding [symbol] |
| 45 | II-22 | GAOGEDGROGPOGPQGARGQOGVMGFO | + | + |
| 46 | II-23 | GQOGVMGFOGPKGANGEOGKAGEKGLO | − | + |
| 47 | III-23 | GPOGPSGPRGQOGVMGFOGPKGNDGAO | + | + |
| 48 | GPS-GFO | GPSGPRGQOGVMGFO | + | + |
| 49 | GPS-GVM | GPSGPRGQOGVM | − | − |
| 50 | GPS-GQO | GPSGPRGQO | − | − |
| 51 | GFOGPK | GFOGPK | − | − |
| 52 | GPR-GPK | GPRGQOGVMGFOGPK | (+)/− | + |
| 3 | GPR-GFO | GPRGQOGVMGFO | + | + |
| 65 | GAR-GFO | GARGQOGVMGFO | + | Not tested |
| 53 | GQO-GFO | GQOGVMGFO | +/− | +/− |
| 5 | GVMGFO | GVMGFO | +/− | +/− |
| 55 | GPS-GFO-5A | GPSGARGQOGVMGFO | + | + |
| 56 | GPS-GFO-6A | GPSGPAGQOGVMGFO | +/− | +/− |
| 57 | GPS-GFO-8A | GPSGPRGAOGVMGFO | + | + |
| 58 | GPS-GFO-9A | GPSGPRGQAGVMGFO | + | + |
| 59 | GPS-GFO-11A | GPSGPRGQOGAMGFO | + | + |
| 60 | GPS-GFO-12A | GPSGPRGQOGVAGFO | − | (+)/− |
| 61 | GPS-GFO-14A | GPSGPRGQOGVMGAO | − | − |
| 62 | GPS-CFO-15A | GPSGPRGQOGVMGFA | (+)/− | +/− |
| 63 | CI-alpha1 | GARGQAGVMGFO | + | + |
| 64 | CI-alpha2 | GARGEOGNIGFO | +/− | (+)/− |
| 54 | GAP-GPR-GFO [symbol] | (GAP) [symbol] -GPRGQOGVMGFO-(GAP) [symbol] | − | − |
| 26 | GFOGER | GFOGER | − | − |
| 66 | II-5 | GARGFOGTOGLOGVKGRRGYOGLDGAK | − | +/− |
| 67 | II- [symbol] 3 | GAKGSAGAOGIAGAOGFOGPRGPOGPQ | − | + |
| 68 | II- [symbol] 9 | GARGAQGPOGATGFOGAAGRVGPOGSN | +/− | +/− |
| 69 | II-44 | GLAGRQGIVGLDGQRGERGFOGLOGPS | − | + |
| 70 | II-46 | GASGDRGPOGPVGPOGLTGPAGEOGRE | +/− | − |
| 71 | III-5 | GERGLOGPOGIKGPAGIDGFOGMKGHR | − | + |
| 72 | III- [symbol] 9 | GPOGPQGVKGERGSDGGDGAAGFOGAR | − | + |
| 41 | III-44 | GAOGPLGIAGITGARGLAGPOGMOGPR | − | + |

[symbol]

DDR1 binding denotes binding to DDR1-Fc or DDR1-Fc2; DDR2 binding denotes binding to DDR2-Fc or DDR2-Fc2, A plus sign (+) indicates a response similar in peptides II-22, III-23, or GPS-GFO (control); a minus sign (−) indicates less than 10% of control; (+)/− indicates 20-30% of control, (+/− indicates 30-80% of control. [symbol]
Data include data from Koniditsiotis et al. (2008) J. Biol. Chem. 283, 6861-6868. [symbol]
GAP-GPR-GFO: non-helical peptide flanked by (GAP) [symbol] instead of (GPP) [symbol] flanking all other peptides. [symbol]
indicates data missing or illegible when filed

In various exemplary embodiments, integrin receptors for different collagens can also be exploited to deliver anti-cancer therapeutic agents using a [(therapeutic agent-linking peptide-integrin targeting peptide)] construct. For example, the collagen-binding A-domains of integrins α1β1 and α2β1 recognize the GFOGER (SEQ ID NO: 26) sequence in native type I and II collagens (Knight et al. 2000). Farndale (2019) summarized various integrin-collagen peptide sequences useful in the present invention. GxOGER (SEQ ID NO: 27) sequences occur in types I, II, and III collagens, while GROGER (SEQ ID NO: 28) occurs in types I and III collagens, but not type II. Different collagen sequences bind different integrins selectively, with α1 and α10β1 having affinity for GLOGEN (SEQ ID NO: 29) in type III collagen, compared with α2 and α11β1 having higher affinity for GFOGER (SEQ ID NO: 26) in types I, II, and IV collagens and others. Integrin α2β1 binds to several GXX′GER (SEQ ID NO: 40) motifs within the collagens, while GXX′GEX″ motifs support binding of all collagen-binding integrins (Farndale et al. 2008). GLPGER (SEQ ID NO: 30), and GFPGER (SEQ ID NO: 31) sequences in streptococcal collagen-like proteins bind al and α2 I-domains in a without requiring hydroxyproline, while GFPGEN (SEQ ID NO: 32) binds only the α1 I-domain (Seo et al. 2010).

IgG-like receptors bind collagen through different motifs. Collagen recognition by GpVI is more complex compared with a21; GPO triplets are essential but not sufficient for binding (Farndale et al. 2008). Binding peptides from collagen toolkits all contain OGPOGP (SEQ ID NO: 33), and two AGPOGP (SEQ ID NO: 34) motifs also contribute to GPVI binding (Feitsma et al. 2022). GPVI is a platelet membrane protein composed of 319 amino acid residues with a molecular weight of 62 kDa (Jung et al. 2008). The structure of the collagen binding domain (CBD) of GPVI is known, and a recombinant GPVI-CBD has been made; similar to related immune receptors, GPVI contains 2 immunoglobulin-like domains arranged in a perpendicular orientation (Horii et al. 2006). LAIR-1, like GpVI, recognizes the GPO triplets found in type I and III collagens, but more complex GPO-containing sequences within the collagens provide their natural ligands (Farndale et al. 2008). LAIR-1, also termed CD305, is a type I glycoprotein of 287 amino acids that was first molecularly cloned in 1997 (Van Laethem et al. 2022). Its structure contains one extracellular C2-type-Ig-like domain and two Immunoreceptor Tyrosine-Based Inhibitory Motif (ITIM) domains in its cytoplasmic tail. The minimal OSCAR binding collagen sequence, identified using an hOSCAR-Fc fusion protein is GPOGPX′GFX' (SEQ ID NO: 35) (Barrow et al. 2011). One such binding peptide is the triple helical (GPO) 3GPOGPAGFO (GPO) 2G (SEQ ID NO: 36), as the triple-helical structure of collagen is critical for OSCAR binding. The three-dimensional structure of the extracellular region of human OSCAR consists of two Ig-like domains, DI (membrane-distal) and D2 (membrane-proximal), connected by a short interdomain linker (Haywood et al. 2016).

In various exemplary embodiments, other targeting peptides can also be used in the [(therapeutic agent-linking peptide-targeting peptide)] construct, depending upon specific tumor cell types and their respective targets. For example, MMP-1 is overexpressed in triple-negative breast cancer (Wang et al. 2019), and GPQG˜LAGQRGIVGLOGQRGER (SEQ ID NO: 37) and GPOGPQG˜LAGQRGIVGLOGQRGER (SEQ ID NO: 38) motifs bind MMP-1 (Farndale 2019). Similarly, MMP-13 is overexpressed in various cancers and plays multiple roles in tumor progression and metastasis (Li et al. 2022), and GPQG˜LAGQRGIVGLOGQRGER (SEQ ID NO: 37) binds MMP-13 (Farndale 2019). Integrin α5β1 is overexpressed in a number of tumors (Hou et al. 2020), as well as novel peptides of sequence RX1ETX2WX3 (SEQ ID NO: 39) bind α5β1 integrin described in U.S. Pat. No. 7,067,619 (incorporated by reference herein in its entirety).

While therapeutic agents can be delivered to a tumor site via tethered peptides that bind directly to cellular receptors on the cancerous cell, the same can also be accomplished via tethered synthetic cellular receptors that bind to peptides motifs in type III collagen devices in various exemplary embodiments. While collagen III devices are preferred based upon their ability to promote a loose collagen structure, the [(therapeutic agent)-(linking peptide)-(synthetic cellular receptor)] conjugate, referred to herein as CONSTRUCT 5, can also be bound to devices fabricated of other of collagen types such as type I collagen, the particular receptor depending upon the particular collagen type. Devices may be in the form of particulate that is directly injected at or near the tumor, or first encapsulated in degradable carrier prior to injection. In various exemplary embodiments, devices may also be in the form of an onlay or a film, surgically implanted at or near the tumor site.

Von Willebrand factor (vWF) binds type III collagen through its A3 domain (Romijn et al. 2003) at a single high-affinity site, GxRGQOGVMGFO (SEQ ID NO: 1) (Farndale 2019), with RGQOGVMGF (SEQ ID NO: 2) being the minimal binding sequence (Lisman et al. 2006). Interestingly, GPRGQOGVMGFO (SEQ ID NO: 3) in type III collagen also binds the DDR2 receptor (Farndale et al. 2008). As such, the [(therapeutic agent)-(linking peptide)-GPRGQOGVMGFO) (SEQ ID NO: 3) conjugate, referred to herein as CONSTRUCT 6, can be either placed directly at or near the site of the tumor, or tethered to collagen III that is subsequently placed at or near the tumor, or both. “Through all of this, the amino acid sequence of collagen molecule contains a myriad of potential interaction sites for ECM ligands, inclusive of DDR, that are maintained consistently through D-periods separated by microns (laterally and axially) (Orgel et al. 2019),” suggesting the use of other as yet undiscovered targeting peptides.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions.

In general, any combination of disclosed features, components and methods described herein is possible. Steps of a method can be performed in any order that is physically possible.

All cited patents, patent publications and non-patent references are incorporated by reference herein in their entireties. Although embodiments have been disclosed, the invention is not limited thereby.

### REFERENCES

Agarwal G. et al. Discoidin domain receptors: Micro insights into macro assemblies. Biochim Biophys Acta Mol Cell Res., 1866(11): 118496 (2019).
Barrow A. D. et al. OSCAR is a collagen receptor that costimulates osteoclastogenesis in DAP12-deficient humans and mice. J Clin Invest., 121(9): 3505-16 (2011).
Boissonnas. A. et al. In vivo imaging of cytotoxic T cell infiltration and elimination of a solid tumor. J Exp Med., 204:345-356 (2007).
Chavda V. P. et al. Peptide-Drug Conjugates: A New Hope for Cancer Management. Molecules., 27 (21): 7232 (2022).
Cortazar P. ct al. US Food and Drug Administration approval overview in metastatic breast cancer. J Clin Oncol., 30(14): 1705-11 (2010).
Di Martino. J. S. et al. A tumor-derived type III collagen-rich ECM niche regulates tumor cell dormancy. Nat Cancer., 3(1). 90-107 (2022).
Ding II. et al. Effectiveness of combination therapy with ISA101 vaccine for the treatment of human papillomavirus-induced cervical cancer. Front Oncol., 12:990877 (2022).
Dhodapkar M. V. et al. Induction of antigen-specific immunity with a vaccine targeting NY-ESO-I to the dendritic cell receptor DEC-205. Sci Transl Med., 6 (232): 232ra51 (2014).
Farndale R. W. et al. Cell-collagen interactions: the use of peptide Toolkits to investigate collagen-receptor interactions. Biochem Soc Trans., 36(Pt 2): 241-50 (2008).
Farndale R. W. Collagen-binding proteins: insights from the Collagen Toolkits. Essays Biochem., 63(3): 337-348 (2019).
Feitsma L. J. et al. Structural insights into collagen binding by platelet receptor glycoprotein VI. Blood., 139(20): 3087-3098 (2022).
Gonzalez H. et al. Roles of the immune system in cancer: from tumor initiation to metastatic progression. Genes Dev., 32(19-20): 1267-1284 (2018).
Harper D. M. et al. The efficacy and safety of Tipapkinogen Sovacivec therapeutic HPV vaccine in cervical intraepithelial neoplasia grades 2 and 3: Randomized controlled phase II trial with 2.5 years of follow-up. Gynecol Oncol., 153(3): 521-529 (2019).
Haywood J. et al. Structural basis of collagen recognition by human osteoclast-associated receptor and design of osteoclastogenesis inhibitors. Proc Natl Acad Sci U SA., 113(4): 1038-43 (2016).
Horii K. et al. Structural basis for platelet collagen responses by the immune-type receptor glycoprotein VI. Blood., 108(3): 936-42 (2006).
Hou J. et al. The Roles of Integrin a5B1 in Human Cancer. Onco Targets Ther., 13:13329-13344 (2020). Hsu K. S. et al. Cancer cell survival depends on collagen uptake into tumor-associated stroma. Nat Commun., 13(1): 7078 (2022).
Jung S. M. et al. Platelet Glycoprotein VI. In: Sigalov, A.B. (eds) Multichain Immune Recognition Receptor Signaling. Advances in Experimental Medicine and Biology, vol 640. Springer, New York, NY. NEED Kanta J. Collagen matrix as a tool in studying fibroblastic cell behavior. Cell Adh Migr., 9(4): 308-16 (2015).
Kim T. J. et al. Clearance of persistent HPV infection and cervical lesion by therapeutic DNA vaccine in CIN3 patients. Nat Commun., 5:5317 (2014).
Knight C. G. et al. The collagen-binding A-domains of integrins α1β1 and α2β1 recognize the amino acid sequence, GFOGER (SEQ ID NO: 26) in native (triple-helical) collagens. J Biol Chem., 275(1): 35-40 (2000).
Konitsiotis A. D. et al. Characterization of high affinity binding motifs for the discoidin domain receptor DDR2 in collagen. J Biol Chem., 283(11): 6861-8 (2008).
Kuczek. D. E. et al. Collagen density regulates the activity of tumor-infiltrating T cells. J Immunother Cancer., 7(1): 68 (2019).
Leitinger B. Discoidin domain receptor functions in physiological and pathological conditions. Int Rev Cell Mol Biol., 310:39-87 (2014).
Li S. et al. Regulation and function of matrix metalloproteinase-13 in cancer progression and metastasis. Cancers (Basel)., 14(13): 3263 (2022).
Lin M. J. et al. Cancer vaccines: the next immunotherapy frontier. Nat Cancer., 3(8): 911-926 (2022). Lisman T. et al. A single high-affinity binding site for von Willebrand factor in collagen III, identified using synthetic triple-helical peptides. Blood., 108 (12): 3753-6 (2006).
Liu. J. et al. New insights into M1/M2 macrophages: key modulators in cancer progression. Cancer Cell Int., 21(1): 389 (2021).
McAuliffe J. et al. Heterologous prime-boost vaccination targeting MAGE-type antigens promotes tumor T-cell infiltration and improves checkpoint blockade therapy. J Immunother Cancer., 9(9): c003218 (2021).
Mittendorf E. A. et al. Primary analysis of a prospective, randomized, single-blinded phase II trial evaluating the HER2 peptide AE37 vaccine in breast cancer patients to prevent recurrence. Ann Oncol., 27(7): 1241-8 (2016).
Mittendorf E. A. et al. Efficacy and safety analysis of Nelipepimut-S Vaccine to prevent breast cancer recurrence: A Randomized, Multicenter, Phase III Clinical Trial. Clin Cancer Res., 25(14): 4248-4254 (2019).
Orgel J. P. R. O. et al. A structural prospective for collagen receptors such as DDR and their binding of the collagen fibril. Biochim Biophys Acta Mol Cell Res., 1866(11): 118478 (2019).
Pandya P. H. et al. The immune system in cancer pathogenesis: potential therapeutic approaches. J Immunol Res., 2016:4273943 (2016).
Romijn R. A. et al. Mapping the collagen-binding site in the von Willebrand factor-A3 domain. J Biol Chem., 278 (17): 15035-9 (2003).
Sawasdikosol S et al. A perspective on HPKI as a novel immuno-oncology drug target. Elife. 9: c55122 (2020).
Sco N. et al. An engineered α1 integrin-binding collagenous sequence. J Biol Chem., 285 (40): 31046-54 (2010).
Si J. et al. Hematopoictic Progenitor Kinasel (HPK1) mediates T cell dysfunction and is a druggable target for T Cell-based immunotherapies. Cancer Cell., 38(4): 551-566 (2020).
Sun. X. et al. Tumour DDR1 promotes collagen fibre alignment to instigate immune exclusion. Nature., 599:673-678 (2021). Tahaghoghi-Hajghorbani S. et al. Targeting the tumor microenvironment by liposomal Epacadostat in combination with liposomal gp 100 vaccine. Sci Rep., 13(1): 5802 (2023).
Takagi J. et al. Structure of integrin a51 in complex with fibronectin. EMBO J., 22(18): 4607-15 (2003).

Taylor G S. Jia H. Harrington K et al. A recombinant modified vaccinia ankara vaccine encoding Epstein-Barr Virus (EBV) target antigens: a phase I trial in UK patients with EBV-positive cancer. Clin Cancer Res., 20(19): 5009-22 (2014).
Van Lacthem F. et al. LAIR1. an ITIM-Containing Receptor Involved in Immune Disorders and in Hematological Neoplasms. Int J Mol Sci., 23(24): 16136 (2022).
Vansteenkiste J. et al. Adjuvant MAGE-A3 immunotherapy in resected non-small-cell lung cancer: phase II randomized study results. J Clin Oncol., 31(19): 2396-403 (2013).
Wang Q. M. et al. MMP-1 is overexpressed in triple-negative breast cancer tissues and the knockdown of MMP-1 expression inhibits tumor cell malignant behaviors in vitro. Oncol Lett., 17(2): 1732-1740 (2019).
Weller M. et al. Rindopepimut with temozolomide for patients with newly diagnosed. EGFRvIII-expressing glioblastoma (ACT IV): a randomised, double-blind, international phase 3 trial. Lancet Oncol., 18(10): 1373-1385 (2017).
Xu. H et al. Collagen binding specificity of the discoidin domain receptors: Binding sites on collagens II and III and molecular determinants for collagen IV recognition by DDR1. Matrix Biol., 30:16-26 (2011).
You D. et al. Enhanced antitumor immunity by a novel small molecule HPK1 inhibitor. J Immunother Cancer., 9: e001402 (2021).
Zeromski J. Significance of tumor-cell receptors in human cancer. Arch Immunol Ther Exp (Warsz)., 50(2): 105-10 (2002).

### WEB REFERENCES

Immune Checkpoint Inhibitors. www.cancer.gov/about-cancer/treatment/types/immunotherapy/checkpoint-inhibitors.
Moderna Product Pipeline. www.modernatx.com/research/product-pipeline. Atezolizumab (Tecentriq).
www.tecentriq.com.
Avelumab (Bavencio). www.bavencio.com/hcp.
Cemiplimab (Libtayo). www.libtayo.com. Durvalumab (Imfinzi). www.imfinzi.com. Enzalutamide
(XTANDI). www.xtandi.com. Ipilimumab (Yervoy). www.yervoy.com.
Nivolumab (Opdivo). www.opdivo.com.
Nivolumab +Relatlimab-rmbw (Opdualag). www.opdualag.com. Pembrolizumab (Keytruda).
www.keytruda.com.
Rituximab (Rituxan). www.rituxan.com.
Tremelimumab (Imjudo). www.accessdata.fda.gov/drugsatfda_docs/label/2022/761289lbl.pdf.

1. A method for treating cancer in a patient, comprising: introducing a collagen device having a DDR1 peptide into the patient, whereby the DDR 1 peptide is delivered to the cancer.

2. The method of claim 1, wherein the collagen device is an injectable device comprising one or more of type III collagen, native rh type III collagen, or rh type III collagen modified with DDR1 binding sites including a DDR1 peptide.

3. The method of claim 1, wherein the collagen device is an onlay device comprising one or more of type III collagen, native rh type III collagen, or rh type III collage modified with DDR1 binding sites including a DDR1 peptide.

4. The method of claim 1, wherein the collagen device is a film comprising one or more of type III collagen, native rh type III collagen, or rh type III collage modified with DDR1 binding sites including a DDR1 peptide.

5. The method of claim 1, wherein the DDR1 peptide includes a DDR1 peptide dispersed in a degradable carrier.

6. The method of claim 5, wherein the carrier is selected from the group consisting of gelatin and other collagens.

7. The method of claim 5, wherein the carrier is a synthetic degradable polymer.

8. The method of claim 5, wherein the carrier is selected from the group consisting of PEG, PLA, PGA, PLGA, and polymers thereof.

9. The method of claim 1, wherein the DDR1 peptide is dispersed in an injectable carrier.

10. The method of claim 1, wherein the DDR1 peptide is selected from the group consisting of
a. a peptide containing the sequence GVMGFO (SEQ ID NO: 5);
b. a peptide containing the sequence GVMGFP (SEQ ID NO: 6);
c. a peptide containing the sequence GPSGFO (SEQ ID NO: 7);
d. a peptide containing the sequence GPSGFP (SEQ ID NO: 8);
e. a peptide containing the sequence GPRGFO (SEQ ID NO: 9);
f. a peptide containing the sequence GPRGFP (SEQ ID NO: 10);
g. a peptide containing the sequence GARGFO (SEQ ID NO: 11);
h. a peptide containing the sequence GARGFP (SEQ ID NO: 12);
i. a peptide containing the sequence GQOGFO (SEQ ID NO: 13);
j. a peptide containing the sequence GQOGFP (SEQ ID NO: 14);
k. a peptide containing the sequence GAMGFO (SEQ ID NO: 15);
l. a peptide containing the sequence GAMGFP (SEQ ID NO: 16);
m. a peptide containing the sequences of one of the peptides a-1 and flanked by at least one amino acid triplet;
n. a peptide sequence that performs a function selected from the group consisting of binding to DDR1, inducing autophosphorylation of cell membrane bound DDR1, and activating a DDR1 signaling pathway;
o. a peptide according to one of the peptides a-n having a form selected from the group consisting of single helical form, double helical form, and triple helical form;
p. a peptide according to one of the peptides a-i that have been modified with positive charged amino acid side chains; and
a peptide according to peptides a-i that have been modified with negatively charged amino acid side chains.

11. The method of claim 10, wherein the at least one amino acid triplet is selected from the group consisting of a GPO amino acid triplet and a GfO amino acid triplet, wherein O is the amino acid hydroxyproline, f is 2S,4S fluoroproline.

12. The method of claim 11, wherein the at least one amino acid triplet is selected from the group consisting of GPO(n)-GVMGFO-GPO(n) (SEQ ID NO: 43) and GfO(n)-GVMGFO-GfO(n) (SEQ ID NO: 44), wherein n is the number of GPO or GfO repeats.

13. A method for treating cancer in a patient, comprising:
introducing a DDR1 peptide into the patient, whereby the DDR1 peptide is delivered to the cancer.

14. The method of claim 13, wherein the DDR1 peptide includes a DDR1 peptide dispersed in a degradable carrier.

15. The method of claim 14, wherein the carrier is selected from the group consisting of gelatin and other collagens.

16. The method of claim 14, wherein the carrier is a synthetic degradable polymer.

17. The method of claim 14, wherein the carrier is selected from the group consisting of PEG, PLA, PGA, PLGA, and polymers thereof.

18. The method of claim 13, wherein the DDR1 peptide is dispersed in an injectable carrier.

19. The method of claim 13, wherein the DDR1 peptide is selected from the group consisting of
a. a peptide containing the sequence GVMGFO (SEQ ID NO: 5);
b a peptide containing the sequence GVMGFP (SEQ ID NO: 6);
c. a peptide containing the sequence GPSGFO (SEQ ID NO: 7);
d. a peptide containing the sequence GPSGFP (SEQ ID NO: 8);
e. a peptide containing the sequence GPRGFO (SEQ ID NO: 9);
f. a peptide containing the sequence GPRGFP (SEQ ID NO: 10);
g. a peptide containing the sequence GARGFO (SEQ ID NO: 11);
h. a peptide containing the sequence GARGFP (SEQ ID NO: 12);
i. a peptide containing the sequence GQOGFO (SEQ ID NO: 13);
j. a peptide containing the sequence GQOGFP (SEQ ID NO: 14);
k a peptide containing the sequence GAMGFO (SEQ ID NO: 15);
l. a peptide containing the sequence GAMGFP (SEQ ID NO: 16);
m. a peptide containing the sequences of one of the peptides a-1 and flanked by at least one amino acid triplet;
n. a peptide sequence that performs a function selected from the group consisting of binding to DDR1, inducing autophosphorylation of cell membrane bound DDR1, and activating a DDR1 signaling pathway;
o. a peptide according to one of the peptides a-n having a form selected from the group consisting of single helical form, double helical form, and triple helical form;
p. a peptide according to one of the peptides a-i that have been modified with positive charged amino acid side chains; and
q. a peptide according to peptides a-i that have been modified with negatively charged amino acid side chains.

20. The method of claim 19, wherein the at least one amino acid triplet is selected from the group consisting of a GPO amino acid triplet and a GfO amino acid triplet, wherein O is the amino acid hydroxyproline, f is 2S,4S fluoroproline.

21. The method of claim 20, wherein the at least one amino acid triplet is selected from the group consisting of GPO(n)-GVMGFO-GPO(n) (SEQ ID NO: 43) and GfO(n)-GVMGFO-GfO(n) (SEQ ID NO: 44), wherein n is the number of GPO or GfO repeats.

22. A method for treating cancer in a patient, comprising:
introducing a DDR1 peptide into the patient, whereby the DDR1 peptide is delivered to the cancer; and
administering an anti-cancer therapeutic agent to the patient.

23. The method of claim 22, where the anti-cancer therapeutic agent is selected from the group consisting of a cancer active pharmaceutical ingredient; a cancer vaccine; and an inhibitor/degrader of HPK1.

24. A method for treating cancer in a patient, comprising:
introducing a collagen device having a DDR1 peptide into the patient, whereby the DDR 1 peptide is delivered to the cancer; and
administering an anti-cancer therapeutic agent to the patient.

25. The method of claim 24, where the anti-cancer therapeutic agent is selected from the group consisting of a cancer active pharmaceutical ingredient, a cancer vaccine, and an inhibitor/degrader of HPK1.


SEQUENCE LISTING (73 sequences)

SEQ ID NO: 1 - protein, 12 aa, synthetic construct
  VARIANT 2: note = X can be any amino acid
GXRGQOGVMGFO

SEQ ID NO: 2 - protein, 9 aa, synthetic construct
RGQOGVMGF

SEQ ID NO: 3 - protein, 12 aa, synthetic construct
GPRGQOGVMGFO

SEQ ID NO: 4 - protein, 16 aa, synthetic construct
YTAIAWVKAFIRKLRK

SEQ ID NO: 5 - protein, 6 aa, synthetic construct
GVMGFO

SEQ ID NO: 6 - protein, 6 aa, synthetic construct
GVMGFP

SEQ ID NO: 7 - protein, 6 aa, synthetic construct
GPSGFO

SEQ ID NO: 8 - protein, 6 aa, synthetic construct
GPSGFP

SEQ ID NO: 9 - protein, 6 aa, synthetic construct
GPRGFO

SEQ ID NO: 10 - protein, 6 aa, synthetic construct
GPRGFP

SEQ ID NO: 11 - protein, 6 aa, synthetic construct
GARGFO

SEQ ID NO: 12 - protein, 6 aa, synthetic construct
GARGFP

SEQ ID NO: 13 - protein, 6 aa, synthetic construct
GQOGFO

SEQ ID NO: 14 - protein, 6 aa, synthetic construct
GQOGFP

SEQ ID NO: 15 - protein, 6 aa, synthetic construct
GAMGFO

SEQ ID NO: 16 - protein, 6 aa, synthetic construct
GAMGFP

SEQ ID NO: 17 - protein, 5 aa, synthetic construct
TGEKP

SEQ ID NO: 18 - protein, 15 aa, synthetic construct
TGGGGSGGGGTGEKP

SEQ ID NO: 19 - protein, 12 aa, synthetic construct
LRQKDGGGSERP

SEQ ID NO: 20 - protein, 9 aa, synthetic construct
LRQKDGERP

SEQ ID NO: 21 - protein, 10 aa, synthetic construct
GGRGRGRGRQ

SEQ ID NO: 22 - protein, 16 aa, synthetic construct
QNKKGGSGDGKKKQHI

SEQ ID NO: 23 - protein, 6 aa, synthetic construct
TGGERP

SEQ ID NO: 24 - protein, 6 aa, synthetic construct
ATGEKP

SEQ ID NO: 25 - protein, 11 aa, synthetic construct
GGGSGGGGEGP

SEQ ID NO: 26 - protein, 6 aa, synthetic construct
GFOGER

SEQ ID NO: 27 - protein, 6 aa, synthetic construct
  VARIANT 2: note = X can be any amino acid
GXOGER

SEQ ID NO: 28 - protein, 6 aa, synthetic construct
GROGER

SEQ ID NO: 29 - protein, 6 aa, synthetic construct
GLOGEN

SEQ ID NO: 30 - protein, 6 aa, synthetic construct
GLPGER

SEQ ID NO: 31 - protein, 6 aa, synthetic construct
GFPGER

SEQ ID NO: 32 - protein, 6 aa, synthetic construct
GFPGEN

SEQ ID NO: 33 - protein, 6 aa, synthetic construct
OGPOGP

SEQ ID NO: 34 - protein, 6 aa, synthetic construct
AGPOGP

SEQ ID NO: 35 - protein, 9 aa, synthetic construct
  VARIANT 6: note = X can be any amino acid
  VARIANT 9: note = X can be any amino acid
GPOGPXGFX

SEQ ID NO: 36 - protein, 25 aa, synthetic construct
GPOGPOGPOGPOGPAGFOGPOGPOG

SEQ ID NO: 37 - protein, 21 aa, synthetic construct
GPQGLAGQRGIVGLOGQRGER

SEQ ID NO: 38 - protein, 24 aa, synthetic construct
GPOGPQGLAGQRGIVGLOGQRGER

SEQ ID NO: 39 - protein, 7 aa, synthetic construct
  VARIANT 2: note = X can be any amino acid
  VARIANT 5: note = X can be any amino acid
  VARIANT 7: note = X can be any amino acid
RXETXWX

SEQ ID NO: 40 - protein, 6 aa, synthetic construct
  VARIANT 2..3: note = X can be any amino acid
GXXGER

SEQ ID NO: 41 - protein, 27 aa, synthetic construct
GAOGPLGIAGITGARGLAGPOGMOGPR

SEQ ID NO: 42 - protein, 18 aa, synthetic construct
  MOD_RES 3: note = Hydroxyproline
  MOD_RES 6: note = Hydroxyproline
  MOD_RES 9: note = Hydroxyproline
  MOD_RES 12: note = Hydroxyproline
  MOD_RES 15: note = Hydroxyproline
  MOD_RES 18: note = Hydroxyproline
GPXGPXGPXGPXGPXGPX

SEQ ID NO: 43 - protein, 12 aa, synthetic construct
  MOD_RES 3: note = Hydroxyproline
  MOD_RES 12: note = Hydroxyproline
  VARIANT 1..3: note = GPX residues can be repeated one or more times
  VARIANT 10..12: note = GPX residues can be repeated one or more times
GPXGVMGFOGPX

SEQ ID NO: 44 - protein, 12 aa, synthetic construct
  MOD_RES 2: note = Fluoroproline
  MOD_RES 11: note = Fluoroproline
  VARIANT 1..3: note = GXO residues can be repeated one or more times
  VARIANT 10..12: note = GXO residues can be repeated one or more times
GXOGVMGFOGXO

SEQ ID NO: 45 - protein, 27 aa, synthetic construct
GAOGEDGROGPOGPQGARGQOGVMGFO

SEQ ID NO: 46 - protein, 27 aa, synthetic construct
GQOGVMGFOGPKGANGEOGKAGEKGLO

SEQ ID NO: 47 - protein, 27 aa, synthetic construct
GPOGPSGPRGQOGVMGFOGPKGNDGAO

SEQ ID NO: 48 - protein, 15 aa, synthetic construct
GPSGPRGQOGVMGFO

SEQ ID NO: 49 - protein, 12 aa, synthetic construct
GPSGPRGQOGVM

SEQ ID NO: 50 - protein, 9 aa, synthetic construct
GPSGPRGQO

SEQ ID NO: 51 - protein, 6 aa, synthetic construct
GFOGPK

SEQ ID NO: 52 - protein, 15 aa, synthetic construct
GPRGQOGVMGFOGPK

SEQ ID NO: 53 - protein, 9 aa, synthetic construct
GQOGVMGFO

SEQ ID NO: 54 - protein, 42 aa, synthetic construct
GAPGAPGAPGAPGAPGPRGQOGVMGFOGAPGAPGAPGAPGAP

SEQ ID NO: 55 - protein, 15 aa, synthetic construct
GPSGARGQOGVMGFO

SEQ ID NO: 56 - protein, 15 aa, synthetic construct
GPSGPAGQOGVMGFO

SEQ ID NO: 57 - protein, 15 aa, synthetic construct
GPSGPRGAOGVMGFO

SEQ ID NO: 58 - protein, 15 aa, synthetic construct
GPSGPRGQAGVMGFO

SEQ ID NO: 59 - protein, 15 aa, synthetic construct
GPSGPRGQOGAMGFO

SEQ ID NO: 60 - protein, 15 aa, synthetic construct
GPSGPRGQOGVAGFO

SEQ ID NO: 61 - protein, 15 aa, synthetic construct
GPSGPRGQOGVMGAO

SEQ ID NO: 62 - protein, 15 aa, synthetic construct
GPSGPRGQOGVMGFA

SEQ ID NO: 63 - protein, 12 aa, synthetic construct
GARGQAGVMGFO

SEQ ID NO: 64 - protein, 12 aa, synthetic construct
GARGEOGNIGFO

SEQ ID NO: 65 - protein, 12 aa, synthetic construct
GARGQOGVMGFO

SEQ ID NO: 66 - protein, 27 aa, synthetic construct
GARGFOGTOGLOGVKGHRGYOGLDGAK

SEQ ID NO: 67 - protein, 27 aa, synthetic construct
GAKGSAGAOGIAGAOGFOGPRGPOGPQ

SEQ ID NO: 68 - protein, 27 aa, synthetic construct
GARGAQGPOGATGFOGAAGRVGPOGSN

SEQ ID NO: 69 - protein, 27 aa, synthetic construct
GLAGQRGIVGLOGQRGERGFOGLOGPS

SEQ ID NO: 70 - protein, 26 aa, synthetic construct
GASGORGPOGPVGPOGLTGPAGOGRE

SEQ ID NO: 71 - protein, 27 aa, synthetic construct
GERGLOGPOGIKGPAGIOGFOGMKGHR

SEQ ID NO: 72 - protein, 27 aa, synthetic construct
GPOGPQGVKGERGSOGGOGAAGFOGAR

SEQ ID NO: 73 - protein, 7 aa, synthetic construct
  VARIANT 1: note = X can be any amino acid
XGEGDGC